(19) United States
(12) Patent Application Publication
(10) Pub. No.: US 2023/0241182 A1
(43) Pub. Date: Aug. 3, 2023

(54) PROTEIN NANOSPHERES TO TREAT HARM FROM MULTIPLE TRAUMA

(71) Applicant: Richard C.K. Yen, Yorba Linda, CA (US)

(72) Inventor: Richard C.K. Yen, Yorba Linda, CA (US)

(21) Appl. No.: 18/295,829

(22) Filed: Apr. 4, 2023

Related U.S. Application Data
(63) Continuation-in-part of application No. 17/208,736, filed on Mar. 22, 2021.
Application No. 17/208,736 is a continuation-in-part of application No. 15/233,779, filed on Aug. 10, 2016, now Pat. No. 11,260,109.
Application No. 15/233,779 is a continuation-in-part of application No. 16/505,257, filed on Jul. 8, 2019, now Pat. No. 11,260,110.
Application No. 16/505,257 is a continuation-in-part of application No. 17/094,114, filed on Nov. 10, 2020.
Application No. 17/094,114 is a continuation-in-part of application No. 17/094,114, filed on Nov. 10, 2020.
Application No. 17/094,114 is a continuation-in-part of application No. 14/953,066, filed on Nov. 27, 2015, now abandoned.
Application No. 14/953,066 is a continuation-in-part of application No. 15/233,779, filed on Aug. 10, 2016, now Pat. No. 11,260,109.
Application No. 15/233,779 is a continuation-in-part of application No. 16/505,257, filed on Jul. 8, 2019, now Pat. No. 11,260,110.
(60) Provisional application No. 63/372,883, filed on Apr. 13, 2022.
(60) Provisional application No. 63/100,670, filed on Mar. 25, 2020.

(51) Int. Cl. A61K 38/38; A61K 9/16; A61P 17/02; B82Y 5/00

(52) U.S. Cl. CPC A61K 38/38; A61K 9/1658; A61P 17/02; B82Y 5/00

(57) ABSTRACT

A product and method of using albumin nanoparticles for treating multiple traumas by augmenting the function or effectiveness of stem cells or precursor cells in vivo. An albumin nanoparticle suspension containing submicron albumin spheres is prepared, with the albumin spheres being capable of augmenting a function and effectiveness of stem cells or precursor cells in vivo for treating multiple traumas at the same time. A predetermined amount of the albumin nanoparticle suspension is administered to a patient before or after an onset of multiple traumas. A function of the stem or precursor cells can be augmented or improved by the albumin spheres to stimulate mobilization toward the traumas, to ameliorate an inflammatory response of the subject by decreasing an amount of RANTES endothelial production on cytokines production, and/or to improve a secretion of fractalkine. The albumin spheres can be bound with fibrinogen molecules in vitro or in vivo.

### CROSS-REFERENCE TO RELATED APPLICATION

This application claims the benefit of priority under 35 U.S.C. § 119(e) based upon co-pending U.S. provisional patent application Ser. No. 63/372,883 filed on Apr. 7, 2022.

This application is a continuation-in-part under 35 U.S.C. § 120 based upon co-pending U.S. non-provisional patent application Ser. No. 17/208,736 filed on Mar. 22, 2021, and co-pending U.S. non-provisional patent application Ser. No. 17/094,114 filed on Nov. 10, 2020.

The entire disclosure of the prior provisional applications are incorporated herein by reference.

### BACKGROUND

### Technical Field

The present technology concerns the mitigation of the harm to a patient caused by multiple traumas which is often more devastating than a sum of an individual trauma.

### Background Description

The treatment of trauma as practiced today is highly dependent on the nature of the trauma. For example, in a car accident, the patient may have a broken bone and some burns. The Emergency Room physician will typically call in experts in orthopedics to treat the bone fracture, and then a general surgeon to treat the burn. If the patient has a co-morbidity such as diabetes, an internist will treat his “glucose levels.” There may be an overall internist or family doctor who will management the “flow of events” or “treatment schedules” to make sure the various treatments are given in a logical and efficient manner, but there is not one kind of specialists today who can deal with multiple traumas or “polytrauma.”

Polytrauma (multitrauma) is a short verbal equivalent used for injured patients usually with associated injury (i.e. two or more severe injuries in at least two areas of the body), less often with a multiple injury (i.e. two or more severe injuries in one body area). A condition for the use of the term polytrauma or multitrauma is the incidence of the traumatic shock and/or hemorrhagic hypotensis and a serious endangering of one or more vital functions of the organism. At least one out of two or more injuries or the sum total of all injuries endangers the life of the injured person with polytrauma. The term “polytraumatism” used in practice may or may not be a synonym of polytrauma, however, it has a direct generalizing relation to it. Polytraumatism embraces the broad health care and general societal problem area relating to severe associated and multiple injuries (i.e. to polytrauma).

It is now known that polytrauma can illicit immune responses from the patient which are very different from the immune response toward single traumas. That is why polytrauma is more likely to be fatal. One should notice that by the time the patient arrives at the hospital, the car accident is already “done”—but unfortunately even though the patient is still breathing at the time he arrives at the hospital, he can soon die. That clearly shows that there is a period of time after the traumatic event (which is a “one-time” event) where a “struggle” is going on in the patient's body. An adequate response (to occur in the body for some duration after the traumatic event) will lead to recover, but an inadequate response will lead to further damage or even demise. All the treatments for trauma today are given to help the patient “tilt the balance” so that the body can mount a response adequate for the insult (the trauma).

It is obvious that the high mortality rate from polytrauma is not only due to the fact that the patient has suffered “many” injuries, but that the sum of treatments (for each of the separate trauma) is still not sufficient to “tilt the balance” towards recovery. In reality, a new type of treatment is needed, quite separate from the treatment conventionally provided for each of the separate traumatic injuries, or even the sum of all the treatments for single-trauma. A new type of treatment is needed to meet the (unexpected) different nature of the response by a patient to polytrauma.

In the field of treatment for trauma, the health provider is aware that there are several separate events which are not always obvious or can be observed immediately. (a) Most trauma or insults to the body are obvious and can be seen immediately, e.g. a car accident which results in a passenger bleeding. However, sometimes the trauma/insult is not apparent, e.g. if a dirty bomb has been exploded and nobody has the equipment to measure radioactivity exposure. Then the focus is on stopping the bleeding and not treatment for irradiation. Exposure to radiation can impede the healing process, if not cause direct harm. By the time the healthy provider is aware that the patient has also been exposed to radiation, the patient may have already been moving into the operating room for surgery. (b) The response of the body is a separate event from the trauma itself. The immediate response of the body may be different from the delayed response. Sometimes the immediate response is exaggerated and can even cause further harm. For example, a bee sting can be an “insult” to the body. But if the patient is allergic to bee sting, or is excessively afraid of bees, the immediate response can lead to far greater long term (or delayed) harm than would occur in another person without these pre-existing “problems.” (c) Regarding treatment, the general public often credit the recovery of the patient to the treatment (or the medicine) prescribed by the doctor. However, in reality, the medical treatment only tilts the response of the body toward recovery—the body still has to do the real biochemistry or cellular process involved in recovery. Since the medical treatment is a separate event than the body's response without treatment, and it is typically given at a time point different from the trauma and the initial response, the kind of treatment to be given should not be “one size fits all” but should ideally be tailored to the need at the time of treatment. For example, if the response to a bee sting is fear, then all the patient needs is sedation and assurance. But if the patient starts to show symptom of anaphylaxis, the treatment can be intramuscular epinephrine.

The treatment of a medical condition is often described by three approaches:
(a) “Prophylaxis” means the treatment is given before the insult is started. Sometime this is not practical, e.g. exposure to irradiation or thermal burn. Very few people would know or can predict when they will get a dose of irradiation or get burnt and how badly.
(b) “Mitigation” means the treatment is given after the insult has occurred. Typically, mitigation is applied so that the harm is less severe than otherwise. Therefore, the treatment is administered soon after the insult event, but before the harm has started or has peaked. In a situation where the patient does not know he has been exposed to injury, the doctor will typically give a mitigative treatment as soon as some signs and symptoms would appear suggesting that the person will get worse unless he is treated. This does not mean that the “disease” or harm will be controlled or cured, it only means the morbidity is less severe. (c)
(c) “Therapy” or therapeutic treatment: this implies that the progression of the harm is stopped or even reversed. Sometimes the degree of help, or the extent to which a treatment really contributes to the recovery is not clear: it may be a condition where the body will recover with or without the treatment. In the case of a first-degree sun burn, the lotion may work mainly to moisturize the skin and decrease the pain or irritation of the skin after excessive sun exposure—the patient will get better anyway, unless he has other problems such as diabetes or poor circulation. But the patient may think he is “cured” or “healed” by the lotion. Fortunately, under our medical system, the efficacy of a product is often evaluated under stringent scientific methods, such as by using a “double-blinded placebo control” approach during clinical trials to make sure the effect on a patient is not just a placebo effect. Even so, the mechanism of action of a given drug or treatment to be administered at a particular time of the disease progression is often not known.

Multiple traumas (i.e. polytrauma) present huge challenges in terms of treatment. In the case of polytrauma, the body may have a distinctly different response than if the person has a single type of trauma. Also, the degree of insult can make a huge difference in terms of recovery. As discussed above, a first degree of burn can be overcome without much medical intervention. A sixth degree of burn is the type that will involve the destruction of bone. It is typically seen in combat situations, e.g. a burn caused by explosives. Even with timely medical interventions, there is almost no hope of the patient ever having recovery to a state like he was before the injury. Therefore, “effective therapy” after polytrauma may have very different meaning than the civilian meaning of this term. It can mean “effective in reducing the damage done” or “effective in allowing the next phase of medical intervention to proceed sooner.” However it may be defined, “effective” will mean resulting in the patient a condition better than having no treatment or having been treated by a less effective method.

In an article published in “J Orthop Surg Res. 2019 Feb. 19; 14(1):58, titled “Burn and thoracic trauma alters fracture healing, systemic inflammation, and leukocyte kinetics in a rat model of polytrauma” the authors (Mangum et al.) made the following statements:

“Singular traumatic insults, such as bone fracture, typically initiate an appropriate immune response necessary to restore the host to pre-insult homeostasis with limited damage to self. However, multiple concurrent insults, such as a combination of fracture, blunt force trauma, and burns (polytrauma), are clinically perceived to result in abnormal immune response leading to inadequate healing and resolution.”

Regarding the use of fibrinogen coated albumin spheres (FAS) for various medical conditions, FAS has been known to have at least two mechanisms of action. (1) the mobilization of stem cells from the bone marrow, to move to a deep wound where the stem cells can differentiate into various functional tissues to complete healing in an accelerated manner. (2) the formation of co-aggregates with activated platelets at a wound site on the endothelium of the blood vessel, to stop bleeding quickly thus resulting in less surgical blood loss.

Yen disclosed the first mechanism in US publication number 20190328844, “Nanoparticles for the therapeutic treatment of radiation-induced ulcers” published on Oct. 31, 2019. Yen disclosed the second mechanism in US publication number 2018002126 “Albumin nanosphere preparations to control bleeding from surgical operations.”, published on Jan. 25, 2018.

One major situation where polytrauma can occur is anticipated by the “Animal Rule.” This Animal Rule was issued by the US government after the 9/11 event so that the U.S. Food and Drug Administration (FDA) can grant pre-approval for use of a treatment or drug for harm such as will occur from a terrorist attack, where radiological or bio-terror or poison modalities are used. The Animal Rule demands that after safety and efficacy are both demonstrated in at least two animal species, clinical trial may proceed in human volunteers to show that (a) the expected dose is safe in uninjured people, and (b) that there is good reason to anticipate that the same or a higher dose is both safe and effective in the injured person.

To highlight the challenges demanded by the Animal Rule, one needs to understand the rational of the standard method of getting marketing approval from the FDA. The standard method has several steps, as follows: (a) demonstration of safety and efficacy of the drug candidate in a relevant animal model, including the lack of adequate effect from a lower dose and no more improvements from a higher dose. Then the “standard dose” recommended by the Sponsor is adopted; (b) demonstration of safety in a human Clinical Trial One, on the safety of the standard dose and higher doses (in case human patients needs higher doses than the animal doses to achieve efficacy); (c) demonstration of the dose that is effective (and still safe) in human patients with a well-defined disease, in Clinical Trial Two; (c) conduct Clinical Trial Three where a large population with other potential issues (e.g. patients with co-morbidities such as diabetics, obesity, old age etc.) with the purpose of finding out when not to use and on what patients this drug must not be used. The reason why human clinical trials have to be performed rigorously even though the drug causes no problem in animals is because human beings can react very differently from animals, depending on the nature of the drug and the disease. After these exhaustive human studies to find out the side effects and potential contraindications, then the FDA may approve the drug to be sold. (d) In addition, the FDA will typically require “post-marketing” surveillance so that in case the drug causes harm that is not discovered in all the clinical trials, the FDA can still stop the drug from being marketed even though is granted an initial approval.

There are several major problems in invoking the Animal Rule when a sponsor asks FDA to approve a drug for use in terrorist attacks. (a) In normal circumstances, there are no patients suffering from a high dose of irradiation or from agents of bacterial warfare. And It is not ethical to harm a volunteer with the potentially lethal doses of irradiation or bacterial infections just so that a sponsor can test if its drug is safe and effective. (b) Even if only a single harming agent is used (e.g. high doses of irradiation) the exposure of different people to the agent is different; therefore, the victims may not form a “homogeneous” population for the study of the efficacy of a drug or therapy; and it may not be ethical to have some injured person to serve as the “control” group because the harm is typically lethal. (c) Even if an animal model is shown to be able to respond to the harmful effects of irradiation or biological warfare (or both), it is impossible to predict what dose of the drug must be given to the human victim, i.e. the human may not respond to the treatment to the same degree as the animals in the animal models. Therefore, there is a strong need for a more reliable approach, which is the use of biological markers where the effective (and still safe) human dose can be reliably predicted from the animal model.

The following Table 1 shows the steps typically needed to show to the FDA that a drug is not only safe and effective in the animal models, but that there is good evidence that the same dose, or a different dose can be given to injured human patients—all before a single harmed patient exists.

Table 1 below illustrates the 4 conditions that will help scientists to plan research work that needs to be done under the Animal Rule, in particular the use of biomarkers. Condition A are healthy animals vs Condition B which are injured animal. In the examples below, we will use a single agent of injury (e.g. high dose of irradiation); if multiple agents (polytrauma) are involved, the situation will obviously be far more complicated. Condition C involves healthy human volunteers. Condition D includes injured human victims: these may exist in the past because of past events, but they are not in existence as a living homogenous population available for study today.

TABLE 1

| | | | INJURIED |
|---|---|---|---|
| | | HEALTHY | PATIENTS |
| | | SUBJECTS | (e.g. after a high |
| SPECIES | SITUATION | (e.g. non-irradiated) | dose of irradiation) |
| Animal Models | Experimental | CONDITION A | CONDITION B |
| Humans | Real life | CONDITION C | CONDITION D |

In the design of animal models, FDA would want to see the “natural history” of harm in a relevant animal model. For example, the response of an animal to a high dose of irradiation (e.g. exposure to a radiation dose equivalent to a person located several miles downwind from an atomic blast) will not be the same as a low dose of irradiation (e.g. the doses used in radiation therapy.) The response of the animal is not expected to be identical to that of human beings. Also the FDA is focused on the indication: treatment for radiation harm to the central nervous system is not the same as treatment for skin ulceration from radiation. Even in the skin model, mini-pigs are preferred over rodents, because rodents heal differently from skin injuries than min-pigs; and mini-pigs' healing process resembles humans a lot better than rodents. However, with enough background data, the scientist can choose the “right species” and obtain enough of the same species of these animals so that when the “experimental conditions” are designed, there is “uniformity” or homogeneity within the group (mini-pigs of the same age, weight and sex). In addition, the harm: i.e. the exposure to a high dose irradiation can be applied to the same area of skin in the body—so that the effect of treatment can be compared with those in the control group.

In the “design” of human trials, there are only the Condition C healthy volunteers. There are no “condition D” patients—and even if they exist, their exposure to harm is not the same, and to different degrees with different agents of harm. Therefore, there is a lack of homogeneity with which to compare treatment vs control (even if the infrastructure of health care exists.)

### SUMMARY

In view of the foregoing disadvantages inherent in the known types of treatment of trauma, the present technology provides a novel protein nanospheres to treat harm from multiple trauma, and overcomes one or more of the mentioned disadvantages and drawbacks of these known compositions, devices or methods. As such, the general purpose of the present technology, which will be described subsequently in greater detail, is to provide a new and novel protein nanospheres to treat harm from multiple trauma and method which has all the advantages of the known compositions and/or methods mentioned heretofore and many novel features that result in a protein nanospheres to treat harm from multiple trauma which is not anticipated, rendered obvious, suggested, or even implied by the known compositions and/or methods, either alone or in any combination thereof.

According to one aspect, the present technology concerns the manufacture of protein nanospheres of medical grade and their use, which includes the intravenous administration of these nanospheres to a patient suffering from a single type of trauma or from multiple types of trauma (polytrauma) at a dose sufficient to increase the survival rate of the patient and decrease the morbidity from any one of the traumas.

According to another aspect, the present technology can include a method of treating multiple traumas in a subject in need thereof. The method can include administering a therapeutically effective amount of a protein nanosphere suspension containing submicron albumin spheres to the subject suffering from multiple types of traumas (polytrauma) at a dose sufficient to increase a survival rate of the subject and decrease a morbidity from any one of the traumas. The albumin spheres can be configured to augment a function or effectiveness of stem cells or precursor cells in vivo to stimulate mobilization of the stem cells or the precursor cells toward the traumas, wherein the traumas are different from each other.

According to yet another aspect, the present technology can include a composition for treating multiple traumas in a subject in need thereof. The composition can comprise a therapeutically effective amount of an albumin nanoparticle suspension containing submicron albumin spheres. The albumin spheres can be configured to augment a function or effectiveness of stem cells or precursor cells in vivo to stimulate mobilization of the stem cells or the precursor cells toward the traumas, wherein the traumas are different from each other.

According to still yet another aspect, the present technology can include a method of increasing concentrations of one or more biological markers (biomarkers) by the administration of nanospheres such as FAS to a healthy subject not suffering from any injury will respond to the administration of the nanospheres by having increased concentrations of the biomarkers where the biomarkers can be mature cells, progenitor cells, stem cells, cell-communication signals such as cytokines, cells with altered oxidative/reductive states, or intra-bone-marrow cells.

According to still another aspect, the present technology can include a method of increasing concentrations of one or more biological markers (biomarkers) by the administration of nanospheres to a healthy subject not suffering from any injury will respond to the administration of the nanospheres such as FAS by having increased concentrations of the biomarkers where the biomarkers are located at their site of origin such as the bone marrow, or endothelium; or at the site of transit from the site of origin to the site of destination, such as blood; or at the site of destination such as an organ like the skin or liver, or a tissue such as muscle or blood vessels.

According to yet another aspect, the present technology can include a method of increasing concentrations of one or more biological markers (biomarkers) by the administration of nanospheres such as FAS to a healthy subject not suffering from any injury will respond to the administration of the nanospheres by having increased concentrations of the biomarkers the concentrations of which can reliably predict the dose of nanospheres which will be effective in the treatment of human beings who may suffer from future injuries where the injuries are irradiation-induced.

According to still yet another aspect, the present technology can include a method of increasing concentrations of one or more biological markers (biomarkers) by the administration of nanospheres to a healthy subject not suffering from any injury will respond to the administration of the nanospheres by having increased concentrations of the biomarkers the concentrations of which can reliably predict the dose of nanospheres which will be effective in the treatment of human beings who may suffer from future injuries where the injuries are caused by polytrauma

In some or all embodiments, the subject is human.

In some or all embodiments, the therapeutically effective amount can be 8 mg/kg or more administered to the subject intravenously.

Some or all embodiments of the present technology can include the step of stimulating a conversion of the stem cells or precursor cells to mature cells.

In some or all embodiments, the albumin spheres of the albumin nanoparticle suspension can be bound with fibrinogen molecules to produce fibrinogen-coated albumin nano-spheres (FAS).

In some or all embodiments, the albumin nanoparticle suspension can include a supernatant configured to maintain osmolarity compatible with blood of the subject.

In some or all embodiments, the supernatant is saline.

In some or all embodiments, the albumin nanoparticle suspension can include a sorbitol solution configured to maintain osmolarity compatible with blood of the subject.

In some or all embodiments, the sorbitol solution can be added to achieve a 5% sorbitol in the suspension.

In some or all embodiments, the albumin nanoparticle suspension can include a sodium caprylate solution.

In some or all embodiments, the multiple types of traumas can be selected from the group consisting of any one or any combination of a surgical wound, an irradiation burn, a burn, a blunt trauma, a bone injury, a chemotherapy agent, a poison, and an irradiation dose.

In some or all embodiments, the multiple types of traumas can be a surgical wound and an irradiation injury, wherein the surgical wound is before or after the irradiation wound.

Some or all embodiments of the present technology can include the step of increasing concentrations of one or more biological markers (biomarkers) by the albumin spheres, wherein the biomarkers can be selected from the group consisting of any one or any combination of the stem cells, a second stem cell different to that of the stem cells, mature cells, progenitor cells, cell-communication signals, cytokines, cells with altered oxidative/reductive states, and intra-bone-marrow cells.

In some or all embodiments, the biomarkers are located at a site selected from the group consisting of a) an origin site being bone marrow or endothelium, b) a transit site from the origin site to a destination site being blood, an organ, a muscle or blood vessels, and c) a destination site being blood, an organ, a muscle or blood vessels.

In some or all embodiments, the concentrations of the biomarkers are configured to predict a dose of the submicron albumin spheres which is effective in a further treatment from future injuries caused by the multiple traumas.

Some or all embodiments of the present technology can include the step of utilizing an increase in monocyte percentages and absolute monocyte counts as biomarkers in blood of the subject to indicate an effectiveness of a dose of the protein nanosphere suspension.

In some or all embodiments, the submicron albumin spheres increases CD34 cells in bone marrow of the subject.

In some or all embodiments, the albumin spheres can be fibrinogen-coated albumin nano-spheres (FAS) that can further ameliorate an inflammatory response of the subject by decreasing an amount of Regulated upon Activation, Normal T Cell Expressed and Presumably Secreted (RANTES) endothelial production on cytokines production, or can improve a secretion of fractalkine (FKN).

There has thus been outlined, rather broadly, features of the present technology in order that the detailed description thereof that follows may be better understood and in order that the present contribution to the art may be better appreciated.

Numerous objects, features and advantages of the present technology will be readily apparent to those of ordinary skill in the art upon a reading of the following detailed description of the present technology, but nonetheless illustrative, embodiments of the present technology when taken in conjunction with the accompanying drawings.

As such, those skilled in the art will appreciate that the conception, upon which this disclosure is based, may readily be utilized as a basis for the designing of other structures, compositions and methods for carrying out the several purposes of the present technology. It is, therefore, that the claims be regarded as including such equivalent constructions insofar as they do not depart from the spirit and scope of the present technology.

It is therefore an object of the present technology to provide a new and novel protein nanospheres to treat harm from multiple trauma that has all of the advantages of the known compositions and/or methods and none of the disadvantages.

It is another object of the present technology to provide a new and novel protein nanospheres to treat harm from multiple trauma that may be easily and efficiently manufactured and marketed.

An even further object of the present technology is to provide a new and novel protein nanospheres to treat harm from multiple trauma that has a low cost of manufacture with regard to both materials and labor, and which accordingly is then susceptible of low prices of sale to the consuming public, thereby making such protein nanospheres to treat harm from multiple trauma economically available to the buying public.

Still another object of the present technology is to provide a new protein nanospheres to treat harm from multiple trauma that provides in the compositions and methods of the known compositions and/or methods some of the advantages thereof, while simultaneously overcoming some of the disadvantages normally associated therewith.

These together with other objects of the present technology, along with the various features of novelty that characterize the present technology, are pointed out with particularity in the claims annexed to and forming a part of this disclosure. For a better understanding of the present technology, its operating advantages and the specific objects attained by its uses, reference should be made to the accompanying drawings and descriptive matter in which there are illustrated embodiments of the present technology. Whilst multiple objects of the present technology have been identified herein, it will be understood that the claimed present technology is not limited to meeting most or all of the objects identified and that some embodiments of the present technology may meet only one such object or none at all.

### BRIEF DESCRIPTION OF THE DRAWINGS

The present technology will be better understood and objects other than those set forth above will become apparent when consideration is given to the following detailed description thereof. Such description makes reference to the annexed drawings wherein:

FIG. 1 is a scanning microscopy image of fibrinogen-coated albumin nanospheres (FAS), being indicated with an arrow, are able to form co-aggregates with activated platelets, being indicated with a star.

FIG. 2A is a microscopy image of immunostaining of an irradiated control group for Fibrinoplate-S (FPS) FPS-promoted stem cells mobilization towards lesion site in radiation-induced skin injuries.

FIG. 2B is a microscopy image of an FPS treated group (anti-CD34 antibody in red and DAPI in blue) for the FPS-promoted stem cells mobilization towards lesion site in radiation-induced skin injuries. DAPI being a fluorescent stain that binds to adenine-thymine-rich regions in DNA.

FIG. 2C is a graphical view of the Red fluorescence intensity for control (diagonal lines) and FPS treated group (crossed lines), *p<0.05 being error bars represent scanning electron microscopy (SEM).

FIG. 3A is a graphical view of FPS as radio-mitigator for skin injuries including rats exposed to 25 Gy locally to the hind leg of were dosed intravenously with Saline (diagonal line) or 8 mg FPS (crossed line) on Days 1 and 2 post-irradiation (*p<0.05 vs Day 0, error bars represent SEM).

FIG. 3B is a graphical view of FPS as a therapeutic treatment for skin injuries including rats exposed as in FIG. 3A were treated with saline (diagonal bars) or FPS (crossed lines) at days 14^{th} and 15^{th} after irradiation, i.e. when lesion appears (or two consecutive days later if lesion appears later). After 9 days the average size of the skin lesion became >200% the lesion at Day 0 for the control group while it became <25% the lesion at Day 0 for the FPS treated group (*p<0.05 vs Day 0, error bars represent SEM).

FIG. 4A is a graphical view of effects of FPS on cytokines production, specifically FPS ameliorates the inflammatory response by decreasing the amount of Regulated upon Activation, Normal T Cell Expressed and Presumably Secreted (RANTES) endothelial production.

FIG. 4B is a graphical view of effects of FPS on cytokines production, specifically FPS improves the secretion of FKN which is important for stem cells development, with diagonal lines representing non-irradiated, dashed diagonal lines representing irradiated control, and crossed lines representing irradiated+FPS (*p<0.05), error bars represent SEM).

FIG. 5 is a graphical view illustrating the test of anti-CD34 and anti-lineage cocktail staining of mouse bone marrow.

FIG. 6 is fluorescence microscope images at 100× magnification using filters for DAPI showing numerous cells identified by the nuclei stained with DAPI and fluorescein isothiocyanate (FITC) labelled drug.

FIG. 7 is fluorescence microscope images at 40× magnification using filters for DAPI showing merged DAPI and FITC.

FIG. 8 is fluorescence microscope images at 40× magnification using filters for DAPI showing numerous cells identified by the nuclei stained with DAPI and FITC labelled drug.

FIG. 9 is a graphical view illustrating flowcytometry profiles.

FIGS. 10A-10D are graphical views illustrating the effect of FPS on bone marrow cells stained for CD34 and lineage.

FIGS. 11A-11D are graphical views illustrating the effect of FPS on cell populations of white blood cells (WBC), lymphocytes (#LYM), monocytes (#MON), granulocytes (#GRA).

FIG. 12 is a fluorescence microscope image of a control rat with the hair completely obstruct the fluorescent image and the legs were shaved in the attempt to distinguish between the autofluorescence and possible drug fluorescence.

FIG. 13 is a fluorescence microscope image of a drug-treated rat #210 (24 hours after fluorescent drug administration). No fluorescence was seen in the leg containing bone marrow (besides the hair autofluorescence interference).

FIG. 14 is a fluorescence microscope image of a drug-treated Rat #212 (72 hours after fluorescent drug administration). No fluorescence was seen in the leg containing bone marrow (besides the hair autofluorescence interference).

The same reference numerals refer to the same parts throughout the various figures.

### DETAILED DESCRIPTION OF THE EMBODIMENTS

While the above-described devices fulfill their respective, particular objectives and requirements, the aforementioned systems, compositions and methods do not describe a protein nanospheres to treat harm from multiple trauma that allows treating of multiple traumas. The present technology additionally overcomes one or more of the disadvantages associated with the known systems, compositions and/or methods.

A need exists for a new and novel protein nanospheres to treat harm from multiple trauma that can be used for treating of multiple traumas. In this regard, the present technology substantially fulfills this need. In this respect, the protein nanospheres to treat harm from multiple trauma according to the present technology substantially departs from the conventional concepts and designs of the known systems, compositions and/or methods, and in doing so provides an apparatus primarily developed for the purpose of treating of multiple traumas.

However, none of the Yen disclosures discuss the use of FAS in the situation of a combination of traumas, i.e. disclosures on surgical blood loss did not discuss whether FAS can be useful in a condition where the patient has surgery in addition to radiation before or after surgery; and disclosures on radiation mitigation, prophylaxis, treatments with FAS did not discuss how to use FAS if the patient also has skin-breaking wounds. Since polytrauma comprising of several kinds of trauma will have produced different sets of cytokines which makes recovery more difficult than a single type of trauma, it is not predictable if FAS can work, nor how FAS should be administered to make it work (in terms of timing, dose, and number of shots over the course of recovery.) Therefore, the present technology of using FAS to reduce the harm of polytrauma, either in administration of FAS before the harm becomes apparent (truly mitigation), or after the harm has become apparent (truly therapeutic) is new and non-apparent from previous disclosures.

In the following description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

Table 2 shows how scientists can use the existence of biological markers (or biomarkers) to provide a basis for the selection of an adequate dose that can be expected to be both safe and effective for the future injured human patient. The present technology can use the example of the concentration of monocytes in the blood as a relevant biomarker. This example shows that a higher dose of the drug (FPS) would have a stronger effect on the concentration of a biomarker. The concentration of monocyte in the healthy rat vs irradiated rat (with or without administration of FPS) has been published. In the published data, Mao et al have shown that in healthy (non-irradiated) rats injected with 8 mg per kg of FPS, there is a statistically significant rise in the concentration of monocytes in the blood. (Mao X W, Nishiyama N, Pecaut M, Gridley D S, Yen R. titled, “Fibrinoplate-S for the Treatment of Radiation-induced Skin Damage”. 61st Radiation Research Society Annual Meeting, Weston, Fla., Sep. 19-23, 2015.)

It is important that the biomarkers can be readily measured in a compartment in the body that is easily accessible and requires the least invasive method to get to (e.g. in the blood and not in the bone marrow which is more invasion to get to.)

An example of the effect of different doses of FPS administered to rats vs humans:

TABLE 2

Concentration of monocytes in
the blood on a given day
(million per mL blood)

| | | | INJURIED |
|---|---|---|---|
| | | | SUBJECTS |
| | | | from more than |
| | Dose | HEALTHY | one agent, i.e. |
| | of FPS | SUBJECTS | POLYTRAUMA |
| | (mg per | HEALTHY | INJURIED |
| | kg) | SUBJECTS | SUBJECTS |
| Rat | 0 | 0.6 | 0.5 |
| | 8 | 1.0 | 0.8 |
| | 16 | 1.2 | 0.9 |
| | 24 | 1.2 | 1.0 |
| Humans | 0 | 0.4 | Not available yet |
| | 8 | 0.8 | Not available yet |
| | 16 | 1.0 | Not available yet |
| | 24 | 1.2 | Not available yet |

In the example above, it can be seen that irradiation in the control rats (administered the same volume of saline and not FPS) resulted in a slight drop in the concentration of the monocyte population from 0.6 to 0.5 million per mL of blood (first row). It is also established that a standard dose of FPS (8 mg per kg) in the healthy rats will raise the concentration of monocytes from 0.6 to 1.0 million per mL (a significant increase) while in the injured group, the rise is from 0.5 to 0.8 million per mL. Here the FDA is probably not concerned about the percentage of improvement (1.0/0.6 in the case of the healthy population vs 0.8/0.5 in the injured group.) This is because the “baseline” of the injured group is lowered (by the irradiation.) The FDA will probably require that the same absolute concentration of the biomarker be achieved by the “anticipated appropriate dose for the injured human person.”

Since independent data on the efficacy of FPS on healing high dose irradiation-induced skin injury in the rat can be achieved by a “standard dose” of 8 mg FPS per kg weight of the animal, it can be noted that at this dose, the biomarker is 0.8 million monocytes per mL of blood (second row).

Turning now to the human volunteer (assuming the highest dose of 24 mg per kg is still very safe), it can be noted that a dose of 16 mg per kg is needed to achieve in the healthy population of humans a biomarker level of 1.0 million per mL of blood. As noted above, the Injured Human population is not available at this time for study. By the time a natural or man-made event actually occurs, there is no appropriate and certified personnel or facility to collect data for the purpose of assessing the effect of the FPS treatment versus control. Therefore, there is a need to use biomarkers to establish a scientific basis for the prediction of the dose which will achieve healing for the injured population which is yet to occur.

From Table 2, it can be seen that a dose of 16 mg per kg of FPS can achieve a monocyte concentration of 1.0 million per mL of blood, which can be achieved by a dose of only 8 mg per kg in the healthy rat. However, FDA may suggest that the effect of irradiation may suppress the monocyte level in the human population more than in the rat population. Therefore, if a dose of 24 mg per kg is still safe in the human being, the dose of 24 mg/kg may be used on the (future) injured person.

If that is the rationale, then after the animal doses are studied for safety and efficacy, with the corresponding concentration of biomarkers known, the clinical trial one will recruit human volunteers who are healthy and the “standard dose” for this indication will be 24 mg per kg. This means some human volunteers may get an even higher dose (e.g. 48 mg per kg, or higher provided there is no excessive fluid overload from the infused volume of fluid) to show that the standard dose is well “bracketed” (with higher and lower doses compared to the standard dose of the drug.)

Therefore, after a biomarker is chosen, it must be studied and measured, in both the healthy animal and injured animal, so that when the clinical trial is started, the human volunteer will not only be protected from unnecessary harm, but that the data from these volunteers will be useful to predict the safe and effective dose for the injured human population.

Now that the rational approach of using biomarkers under the Animal Rule is understood, we can use the same rational to manage the “more manageable” human population suffering from polytrauma. One example of a more manageable polytrauma case is the cancer patient who needs surgery and radiation therapy within a short time, with the expectation that each of the treatments may retard the recovery from the other, or both. It should be noted that the cancer patient might have recently received chemotherapay also, which is a form of “toxin” even though the chemotherapy is aimed mainly at cancer cells. This situation is still considered “more manageable” because we do have patients who fulfill the criteria of facing these two or more “agents of harm” from whom we can gain experience on how the drug should be used. In these cases, the response of the patients to various doses of FPS can be mapped (i.e. the condition D can be studied, with respect to biomarker levels, which can be correlated later to actual clinical conditions of healing.)

With additional actual data correlating the human dose to the level of biomarker, and to the eventual clinical outcome, the sponsor can show FDA with a greater degree of confidence based on experience and science, on the proper use of the drug. This will hopefully lead to FDA approval for use of the drug on the harder cases of polytrauma (to be discussed below.)

Table 3 below, however, concerns polytrauma where it is harder to get a “homogenous” population of human patients for study. Again, even with many “more manageable cases” there are issues, including (a) when a polytrauma event occurs, different people get exposed to different harms in a different way, and their responses are not the same. For example, in a car accident where the victims suffer burns, have broken bones, and receive blunt trauma, the bones are different, and the organs suffering the most blunt trauma are not the same. Therefore, there is heterogeneity within the “group.” (b) it is hard to obtain “informed consent” after the incident, when the victims are either unconscious or under medical sedition. Therefore, the use of biomarkers would be most helpful to guide the use of a new drug or therapeutics such as disclosed in this patent application.

Table 3 shows an example of different dose of FPS resulting in different response of the biomarker Fraktalkine: the polytrauma for example is the combination of surgery and radiation therapy.

TABLE 3

| | | The maximal concentration Fraktalkine in the blood | |
|---|---|---|---|
| | | during treatment (ph per mL blood) | |
| | | | INJURIED |
| | Dose | | SUBJECTS |
| | of FPS | | from more than |
| | (mg per | HEALTHY | one agent, i.e. |
| | kg) | SUBJECTS | POLYTRAUMA |
| Rat | 0 | 85 | 70 |
| | 8 | 125 | 105 |
| | 16 | 135 | 110 |
| | 24 | 140 | 120 |
| Humans | 0 | 65 | Not available yet |
| | 8 | 90 | Not available yet |
| | 16 | 100 | Not available yet |
| | 24 | 110 | Not available yet |

In the example of the biomarker Fraktalkine, in the saline-treated (no FPS) rats, polytrauma caused a drop of this biomarker in the healthy rats from 85 units (ph per mL blood) to 70 units (first row). Since the therapeutic dose in the rat for the combined injury of surgery and radiation is 8 mg FPS per kg, and the level of Fraktalkine in the healthy rat is 125 units vs 105 units in the polytrauma rats, the aim for the human polytrauma patient should be at least 105 units. Since the healthy rats always have a Fraktalkine level higher than in the polytrauma rats (for every dose level of FPS), it is probably not enough to administer a dose of 16 mg FPS per kg in the human population to treat polytrauma, because at this dose, the healthy human population only generates a level of the biomarker at 100 units. The starting point will be 24 mg FPS per kg weight of the patient, with the hope that in the (future) human patient with the same kind of polytrauma, the “therapeutic” level of 105 units may be reached. Again, it is entirely possible that the sponsor would try a higher dose, e.g. 32 mg FPS per kg, provided the clinical trials in healthy human volunteers can show that this higher dose of FPS is still safe.

In conclusion, in the absence of a well-defined population of human patients suffering from polytrauma, the use of biomarkers from healthy animals vs polytrauma animals provides the best scientific guidance on how to choose an appropriate dose that is best suited for use in the human population in the event that similar polytrauma occurs and the conventional method of getting FDA approval is not possible.

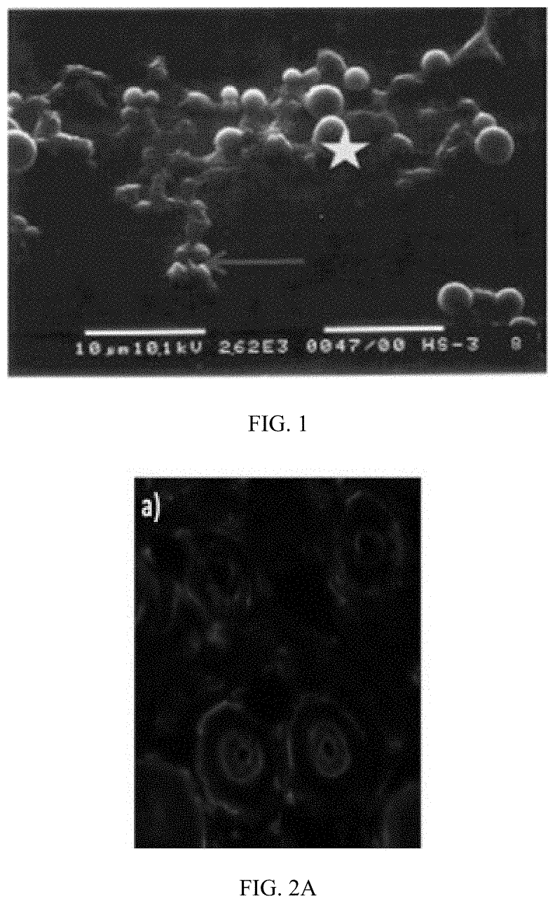

Referring now to the drawings, and particularly to FIGS. 1-4B, an embodiment of the protein nanospheres to treat harm from multiple trauma of the present technology is shown with a description thereof provided herewith.

While certain numbers, values, concentrations are provided in this disclosure, it should be understood that these are only examples and only a small number of variations available to the practice of the present technology. Anyone skilled in the art may attempt to use other values, numbers, or concentrations to circumvent the present technology, but they are all within the scope and spirit of the present technology.

It has been found according to one or more aspects of the present technology that the administration of nanospheres to a subject suffering from a single trauma such as a skin wound from a surgical cut will result in faster recovery and more complete recovery than control subjects treated with normal saline.

It has been found according to one or more aspects of the present technology that the administration of nanospheres to a subject suffering from a single trauma such as a high dose of irradiation to the skin will result in faster recovery and more complete recovery than control subjects treated with normal saline.

It has been found according to one or more aspects of the present technology that the administration of nanospheres to a subject suffering from more than a single trauma such as a surgical wound, in combination with irradiation injury, whether the irradiation is before or after the surgical wound, will result in faster recovery and more complete recovery of either kind of wound than control subjects treated with normal saline.

It has been found according to one or more aspects of the present technology that the administration of nanospheres to a subject suffering from more than a single trauma such as a surgical wound, in combination with irradiation injury, whether the surgical wound is before or after the irradiation wound, will result in faster recovery and more complete recovery of either kind of wound than control subjects treated with normal saline.

It has been found according to one or more aspects of the present technology that the administration of nanospheres to a subject suffering from polytrauma such as a combination of (a) burn, (b) blunt trauma to the chest, (c) bone injury, will result in faster recovery and more complete recovery in the burn injury than control subjects which had been treated with normal saline or the best available treatment for burn.

It has been found according to one or more aspects of the present technology that the administration of nanospheres to a subject suffering from polytrauma such as a combination of (a) burn, (b) blunt trauma to the chest, (c) bone injury, will result in faster recovery and more complete recovery in the blunt trauma injury than control subjects which had been treated with normal saline or the best available treatment for blunt trauma.

It has been found according to one or more aspects of the present technology that the administration of nanospheres to a subject suffering from polytrauma such as a combination of (a) burn, (b) blunt trauma to the chest, (c) bone injury, will result in faster recovery and more complete recovery in the bone injury than control subjects which had been treated with normal saline or the best available treatment for bone injury.

It has been found according to one or more aspects of the present technology that the administration of nanospheres to a subject suffering from polytrauma such as a combination of (a) burn, (b) blunt trauma to the chest, (c) bone injury, will result in faster recovery and more complete recovery in at least one of the three injuries than control subjects which had been treated with normal saline or the best available treatment for burn, blunt trauma and bone injury.

It has been found according to one or more aspects of the present technology that the administration of nanospheres to a subject suffering from polytrauma such as a combination of (a) burn, (b) blunt trauma to the chest, (c) bone injury, (d) extreme high doses of local or whole body irradiation such as from a nuclear blast (from war or from a nuclear energy plant accident), (e) a chemotherapy agent, (f) a poison will result in faster recovery and more complete recovery and improved survival rate from any of the injuries from the polytrauma, when compared to control subjects which had been treated with normal saline or the best available treatment for any one of the injuries caused by the polytrauma.

It has been found according to one or more aspects of the present technology that the administration of nanospheres to a healthy subject not suffering from any injury will respond to the administration of the nanospheres by having increased concentrations of certain biological markers (biomarkers) where the biomarkers can be mature cells, progenitor cells, stem cells, cell-communication signals such as cytokines, cells with altered oxidative/reductive states, or intra-bone-marrow cells.

It has been found according to one or more aspects of the present technology that the administration of nanospheres to a healthy subject not suffering from any injury will respond to the administration of the nanospheres by having increased concentrations of certain biological markers (biomarkers) where the biomarkers are located at their site of origin, or at the site of transit from the site of origin to the site of destination, or at the site of destination such as an organ like the skin or liver, or a tissue such as muscle or blood vessels.

It has been found according to one or more aspects of the present technology that the administration of nanospheres to a healthy subject not suffering from any injury will respond to the administration of the nanospheres by having increased concentrations of certain biological markers (biomarkers) the concentrations of which can reliably predict the dose of nanospheres which will be effective in the treatment of human beings who may suffer from future injuries where the injuries are irradiation-induced.

It has been found according to one or more aspects of the present technology that the administration of nanospheres to a healthy subject not suffering from any injury will respond to the administration of the nanospheres by having increased concentrations of certain biological markers (biomarkers) the concentrations of which can reliably predict the dose of nanospheres which will be effective in the treatment of human beings who may suffer from future injuries where the injuries are caused by polytrauma.

It has been found according to one or more aspects of the present technology that the administration of a sufficient dose of nanospheres to a patient suffering from any one of the injuries listed in the polytrauma cases will respond to the administration of this dose of nanospheres by having increased concentrations of certain biological markers (biomarkers) the concentrations of which can indicate that this dose of nanospheres will be effective in promoting the recovery of the injured person.

It has been found according to one or more aspects of the present technology that the administration of a sufficient dose of nanospheres to a patient suffering from any one of the injuries listed in the polytrauma cases will respond to the administration of this dose of nanospheres by having increased concentrations of certain biological markers (biomarkers) and having an accelerated rate of recovery from the injury.

It has been found according to one or more aspects of the present technology that the administration of a sufficient dose of nanospheres to a patient suffering from any combination of the injuries listed in the polytrauma cases will respond to the administration of this dose of nanospheres by having increased concentrations of certain biological markers (biomarkers) the concentrations of which can indicate that this dose of nanospheres will be effective in promoting the recovery of the injured person.

It has been found according to one or more aspects of the present technology that the administration of a sufficient dose of nanospheres to a patient suffering from any combination of the injuries listed in the polytrauma cases will respond to the administration of this dose of nanospheres by having increased concentrations of certain biological markers (biomarkers) and having an accelerated rate of recovery from any one of the injury or a combination of the injuries.

It has been found according to one or more aspects of the present technology can include a method of treating multiple traumas in a subject in need thereof. The method can include administering a therapeutically effective amount of a protein nanosphere suspension containing submicron albumin spheres to the subject suffering from multiple types of traumas (polytrauma) at a dose sufficient to increase a survival rate of the subject and decrease a morbidity from any one of the traumas. The albumin spheres can be configured to augment a function or effectiveness of stem cells or precursor cells in vivo to stimulate mobilization of the stem cells or the precursor cells toward the traumas, wherein the traumas are different from each other.

It has been found according to one or more aspects of the present technology can include a composition for treating multiple traumas in a subject in need thereof. The composition can comprise a therapeutically effective amount of an albumin nanoparticle suspension containing submicron albumin spheres. The albumin spheres can be configured to augment a function or effectiveness of stem cells or precursor cells in vivo to stimulate mobilization of the stem cells or the precursor cells toward the traumas, wherein the traumas are different from each other.

In some or all embodiments, the subject is human.

In some or all embodiments, the therapeutically effective amount can be 8 mg/kg or more administered to the subject intravenously.

Some or all embodiments of the present technology can include the step of stimulating a conversion of the stem cells or precursor cells to mature cells.

In some or all embodiments, the albumin spheres of the albumin nanoparticle suspension can be bound with fibrinogen molecules to produce fibrinogen-coated albumin nano-spheres (FAS).

In some or all embodiments, the albumin nanoparticle suspension can include a supernatant configured to maintain osmolarity compatible with blood of the subject.

In some or all embodiments, the supernatant can be any one of or any combination of saline, a sorbitol solution, and a sodium caprylate solution.

In some or all embodiments, the sorbitol solution can be added to achieve a final concentration of up to 5% sorbitol in the suspension.

In some or all embodiments, the albumin nanoparticle suspension can include a sodium caprylate solution.

In some or all embodiments, the multiple types of traumas can be selected from the group consisting of any one or any combination of a surgical wound, an irradiation burn, a burn, a blunt trauma, a bone injury, a chemotherapy agent, a poison, and an irradiation dose.

In some or all embodiments, the multiple types of traumas can be a surgical wound and an irradiation injury, wherein the surgical wound is before or after the irradiation wound.

Some or all embodiments of the present technology can include the step of increasing concentrations of one or more biological markers (biomarkers) by the albumin spheres, wherein the biomarkers can be selected from the group consisting of any one or any combination of the stem cells, a second stem cell different to that of the stem cells, mature cells, progenitor cells, cell-communication signals, cytokines, cells with altered oxidative/reductive states, and intra-bone-marrow cells.

In some or all embodiments, the albumin spheres can be fibrinogen-coated albumin nano-spheres (FAS) that can further ameliorate an inflammatory response of the subject by decreasing an amount of Regulated upon Activation, Normal T Cell Expressed and Presumably Secreted (RANTES) endothelial production on cytokines production, or can improve a secretion of fractalkine (FKN).

### EXPERIMENTAL DETAILS AND RESEARCH STRATEGY

### Research Strategy

### 1. Significance

Impaired wound healing after radiation therapy is a concern for the management of cancer patients. Over 60% of cancer patients receive radiotherapy at some point during their course of treatment¹. Ionizing radiation is usually used along with surgical resection in the treatment of solid tumors to shrink the tumor before surgical procedure or decrease local recurrence after surgery. Although effective in treating many cancers, irradiation can result in injury to overlying skin² and disruption of the biological pathways underlying wound healing^{3,4}, leading to higher rates of surgical morbidity, including poor wound healing, chronic ulceration and infections⁵.

Current clinical practice is mainly focused on the conservative management of radiogenic ulcers. Traditional wound care based on polymers including collagen, silicon, chitosan, and hyaluronic acid are commonly used for acute radiodermatitis. For persistent wounds, reconstructive surgery from skin grafts is performed but is often limited by poor surrounding tissue quality and recipient vasculature⁶. Mechanical options like compression bandages and intermittent pneumatic compression are available to treat various venous insufficiency^{7,8}. While, vacuum assistant closure and hyperbaric oxygen chamber are used to improve local oxygenation and blood flow^{9,10}. However, these options are expensive, invasive, and not suitable for all patients. Alternative approaches based on pharmaceutical drugs to improve capillary microcirculation such (e.g. Pentoxifylline) or systemic corticosteroids used to lower the inflammatory response¹¹ don't have tangible effect on wound healing and have potential side effects, deleterious for frail cancer patients^{12,13}.

The lack of approaches targeting the pathogenesis of impaired wound healing³ after radiation constitutes a major surgical difficulty. When radiotherapy treatments are given less than 3 weeks before surgery, a significant increase in wound complications results¹⁴. On the other hand, because radiotherapy delays wound healing, its administration after surgical intervention may be delayed by up to several weeks to allow first complete healing of surgical wounds, with potential negative outcomes on cancer remission¹⁵.

Some or all embodiments of the present technology can include an intravenous (i.v.) injectable biological product that could be safely administered to support the wound healing in irradiated tissues, potentially improving management of cancer patients.

### Scientific Premise

Radiation disrupts the normal process of wound healing³ with pathologic changes including cellular depletion, extracellular matrix changes, microvascular damage, and cytokine and growth factor dysregulation. Radiation has been shown to cause the overexpression of pro-inflammatory cytokines including the Tumor Necrosis Factor-α (TNF-α), Interferon-γ (IFN-γ), Interleukin-1 and Interleukin-6¹⁶. This alteration sustains the inflammatory stage of healing and prevent collagen deposition leading to a decreased wound strength. Upregulated pro-inflammatory cytokines also activate macrophages and stromal cells to produce Transforming Growth Factor-beta (TGF-β1), resulting in fibrosis¹⁷. At the same time, a decrease in Nitric Oxide (NO)¹⁸ and vascular mediators such as the angiogenic growth factors VEGF and bFGF have been shown to induce microvascular damage and tissue hypoxia^{19,5,20}.

Research into new therapeutic approaches that could target the pathogenesis of impaired wound healing after radiation has been so far focused on special dressings, topical administration of active substances and injection of (multipotent) cells. Hydrogel membranes have been proposed as dressing to maintain a moist environment over the wound bed and promote re-epithelialization through the accumulation of cytokines and growth factors²¹ but their treatment effect in radiogenic wounds has not been demonstrated. Topical application of active agents including Histone deacetylase inhibitors²², TGF-beta²³ or recombinant human epidermal growth factor (rhEGF) have not yet provided conclusive results. Several investigators examined the use of mesenchymal or adipose stem cells for repletion and prevention of cell damage and apoptosis. The use of multipotent adult stem cells is an attractive choice due to their large proliferative potential, their proangiogenic²⁴ and antioxidant effects²⁵, as well as their potency to synthesize growth factors and cytokines^{26,27}. Despite the promising application, the use in clinical practice is still far to be reached, as only modest improvements in the length of time to closure and breaking strength²⁸. Moreover, the risk of inducing neoplastic lesions by injecting viable cells as well as logistics challenges in the use of cell therapy still limit the adoption of this approach.

Remarkably none of the approaches described have been validated to support healing of surgical wound associated with radiotherapy. A non-invasive solution able to support surgical wound healing in radiotherapy patients remain an unmet clinical need.

### Proposed Solution

### Fibrinoplate-S (the Drug Product, Containing the Drug Substance Called Fibrinogen-Coated Albumin Spheres, FAS) is a Supportive Care Treatment for Irradiated Patients.

Some or all embodiments of the present technology can include Fibrinoplate-S (FPS), an intravenous (i.v.) injectable first-in-class biological product based on Fibrinogen-coated Albumin nano-Spheres (FAS). Some or all embodiments of the present technology can include FPS as a medical countermeasure (MCM) for nuclear emergency scenarios to mitigate and treat the effects of exposure to high ionizing radiation on wounds. FPS's efficacy is due to its tissue-regeneration properties. FPS can stimulate the mobilization of different lineages of endogenous progenitor cells, including Endothelial Progenitor Cells (EPCs), which are essential to the formation of new tissues²⁹. FPS has also proven to reduce the inflammatory state of radiation wounds by regulating the overexpressed cytokines profile. Together with its broad spectrum of activity, FPS shows extreme ease of use (administered via intravenous injection), long shelf life (˜3 years) and safety (no side effects observed in animals at the effective dose). Some or all embodiments of the present technology proposes to investigate the feasibility of using FPS to support wound healing in oncologic patients undergoing pre- or post-surgical radiotherapy treatment.

### Impact of the Project

### Shift in Clinical Practice.

The proposed solution will have a great impact on the clinical management of oncologic patients. FPS is a simple and safe solution, perfect for those patients already weakened by cancer. Administered straight after a radiotherapy session, the solution could support the outcomes of surgical intervention, promoting prompt wound healing. The solution would be available for both surgeons and radiation oncologists who share a common domain of responsibility towards the management of cancer patients. FPS has the potential to become a new supportive standard of care to treat the health-related side effects of pre- or post-surgical radiotherapy, reducing the cost of hospitalization and improving the quality of life of patients. FPS will be also available as treatment for radiation-induced skin injuries patients commonly resulting from Radiotherapy sessions.

### Contribution to Scientific Knowledge.

Fibrinoplate-S (FPS) offers a therapeutic strategy aimed to improve the clinical management of the multitude of cancer patients in the US, falling with the mission of the National Cancer Institute (NCI). By illustrating the mode of action of the product and its effects on wound recovery, FPS can provide a better understanding of the biological pathways behind wound healing and how the biomarkers are involved and connected in the complex context of wound repair.

### 2. Innovation

Fibrinoplate-S (FPS) is a suspension of Fibrinogen-coated Albumin nano-Spheres (FAS) that promotes accelerated wound healing and coordinated self-repair of multiple tissues by promoting the mobilization of Endothelial Progenitor Cells. Nanometer-sized spheres (diameter: 100-200 nm) are synthesized entirely from sterile material under aseptic conditions in a controlled environment (GMP certified facility), using a proprietary production technique^{34,35}. The starting material to produce FAS is albumin, representing up to 60% of blood plasma proteins and, therefore, it is fully compatible with human blood. Once coated with fibrinogen, the nano-spheres have a median diameter <0.5 μm, (<1% are >1 μm, no sphere is >5 μm) and are 10 times smaller than natural platelets (˜2 μm), so they cannot obstruct even the smallest blood vessels (capillaries' diameter: ˜7 μm).

Some of the innovative features of FAS can be summarized as follows:
High shelf life & easy storage: FAS can be stored for over one year at room temperature, with no special equipment needed; it is contained into simple vials, so it can be easily transported;
Easy administration: FAS is readily injectable intravenous;
Long bioavailability: Studies demonstrated the availability of FAS in the body even after 5 days³⁴;
Safety: FAS is safe, and the absence of toxic reactions was confirmed by extensive preliminary work on irradiated mice (doses as high as 24 mg of FAS spheres per kg weight);
Sterile GMP production: FAS is entirely manufactured from clinical grades of albumin and fibrinogen in a controlled environment, with no risk of bacterial or other infectious agent contamination.

### In-House Medical-Grade Fibrinogen Production.

Non-medical grade fibrinogen is expensive (>1,000 $/gram), and not suitable for human; while medical-grade fibrinogen is even more expensive, and not easily available on the market. Some or all embodiments of the present technology can include a new process to easily obtain large quantities of medical grade and sterile fibrinogen in their own GMP facilities. It is extracted aseptically from clinical grades of fresh-frozen plasma derived from healthy donors and specially prepared in a buffer compatible with the blank sphere suspension with high alcohol content (above 50% v/v) in order to coat the spheres without causing precipitation of the spheres.

### FPS Promotes Coordinated Multiple-Tissue Regeneration.

FPS was originally developed to improve the hemostatic capacity of thrombocytopenic patients. FPS works by forming co-aggregates with platelets to serve as platelet plugs at wound sites on the walls of the blood vessels where the patient's own residual platelets are being activated to form platelet plugs³⁶. However, work in Radiation Skin Injuries (RSI) animal models and subsequent independent propriety work revealed that FPS shows a stimulatory effect on (a) stem cells, including CD34+ cells in the bone marrow, and (b) progenitor cells population of various lineages: from Hematopoietic Progenitor Cells (HPCs), responsible of white blood cells production, to Endothelial Progenitor Cells (EPCs), able to promote accelerated wound healing and coordinated self-repair of multiple tissues in ulcerated-necrotic tissues³⁵.

### Mode of Action.

1—Coordinated self-repair of multiple tissues. When the vascular system is fatally compromised in ulcerated, necrotic tissues, FPS can trigger accelerated healing of the damaged tissues by leveraging the stimulation and mobilization of progenitor cells (see preliminary data). FPS is able to mobilize different lineages of progenitor cells, including Endothelial Progenitor Cells (EPCs), a type of precursor cells located on the endothelium³⁷. While circulating in the vascular system, fibrinogen carried by the albumin nanospheres can interact with GlycoProtein IIb/IIIa (GPIIb/IIIa) receptors, typically present on the surface of platelets and progenitor stem cells. The expression of GPIIb/IIIa receptor has been repeatedly shown, among others, on the surface of hematopoietic progenitor cells, eventually differentiating in the erythroid and myeloid lineages, and also into lymphocytes^{36,38}. Thanks to its nanometer size, FAS nanospheres circulate rheologically near to the endothelium of the vascular system, distributing in arteries, veins, and capillaries, including sinusoids. Thus, the rheological properties of FPS allow direct stimulation of progenitor cells by interaction with their surface receptors. Published medical literature has shown the importance of stem cells moving from the bone marrow and the endothelium to a wound to transform directly or indirectly into the “end-tissue”, e.g. tissues at the site of a wound^{39,40}.

2—Regulation of cytokines profile. FPS support The inflammatory and proliferative phases of wound healing, disrupted by radiation, by regulating the cytokines profile (see preliminary data).

3—Increased white blood cell population. The mobilization of progenitor cells by means of FPS results in an increase total White Blood Cell (WBC) count and of its subgroups (lymphocytes, Monocytes, Granulocytes), offering protection against wound infections.

4—Augmentation of hemostatic functions. When the patient's own thrombin is activated (e.g. in case of lacerated endothelium), the fibrinogen carried on the FAS spheres reacts by forming FAS co-aggregates which include also the residual endogenous platelets. As shown in FIG. 1, when associated with activated platelets, FPS provides an additional mass (i.e. the albumin spheres) to augment the formation of a wound-sealing clot, with the FAS being indicated with a red arrow are able to form co-aggregates with activated platelets indicated with a yellow star. However, FAS do not form co-aggregates among themselves even after interaction with thrombin (possibly due to stereo-hindrance from the bulk of the spheres). Instead, they only form cross-linking masses when free fibrinogen (i.e. fibrinogen not attached to spheres) is present in the solution. In vivo, at the site of a vascular injury, exposed basement membrane collagen causes platelet adherence and platelet activation. The initial platelet plug leads to the local formation of thrombin from soluble prothrombin molecules in the plasma and cleavage of fibrinogen into fibrin. The sub-micron size of the FAS concentrates it near the endothelium (due to rheological effects). Localization to the endothelium allows FAS to be passively entrapped by the activated platelets to form a therapeutic clot such that the effects are localized to the wound site and not disseminated intravascularly. This property guarantees hemostatic functions even if patients' natural platelet count is too low.

### 3. Approach

### Preliminary Studies

The extensive preclinical studies conducted by some or all embodiments of the present technology on the efficacy of FPS on radiation-induced skin injuries demonstrated the benefits on several biological pathways, which are involved in impaired wound healing, supporting the use of FPS in irradiated oncologic patients undergoing surgical procedures.

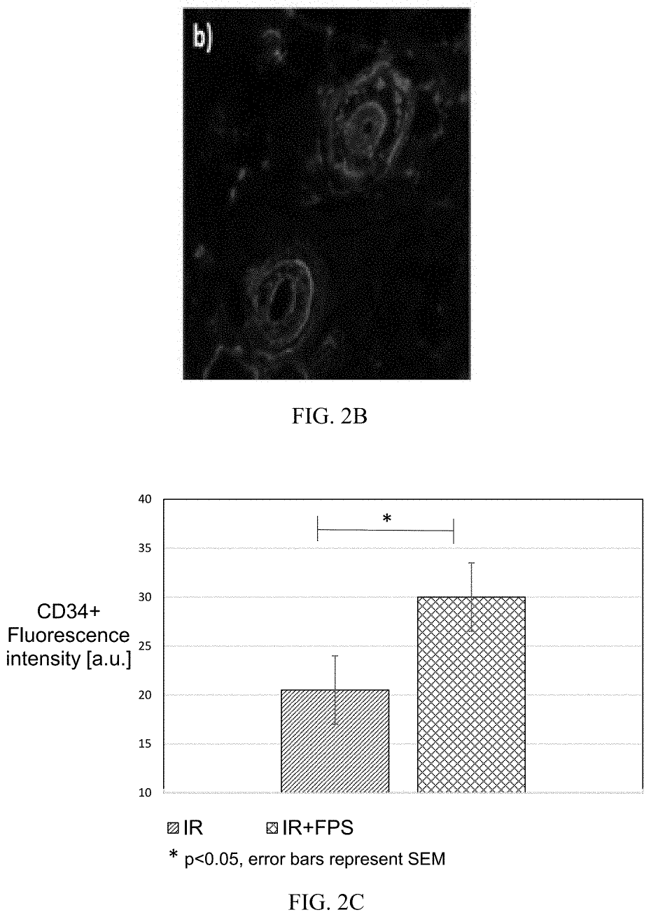

1—FPS is an effective medical countermeasure for the treatment of Radiation Skin Injuries. FPS was first validated as a radio-mitigator for RSI. FPS promoted the coordinated healing of multiple tissues in deep-tissue ulcers, including fat, muscle, nerve and blood vessels in rat model of Radiation Skin Injuries (RSI). Rats treated intravenously (iv) with FAS (8 mg/kg) 1 and 2 days post-irradiation show better healing of localized skin lesions (hind leg locally exposed to 25 Gy) compared to control saline-treated rats (FIG. 2A). On Day 50 after radiation, the lesions of FPS treated group was found more than 50% reduced compared to untreated rats. These results supported significantly accelerated wound healing in ulcerated-necrotic tissues in RSI. Some or all embodiments of the present technology also demonstrated that FPS is an effective therapeutic treatment for radiation skin injuries after skin injury has appeared. To do so, a dose of 25 Gy was delivered locally to the hind leg of rats. Rats were treated intravenously with either FPS (8 mg/kg) or saline, after the appearance of the lesion (FIG. 2B). After 9 days from treatment, the FPS treated group experienced a significant reduction of the lesion size down to 0.6±0.2 cm2, <25% of the initial size on Day 0 (FIG. 2B, green, p<0.05), confirming the ability of FPS to promote healing of established skin lesions. During the 70 days post-treatment, FPS did not show toxicity or severe adverse effects, and the treated group only experienced non-significant weight loss (>10%) compared to the control group (data not shown).

FIG. 2C is a graphical view of the red fluorescence intensity for control (shown with diagonal lines) and FPS treated group (shown with crossed lines).

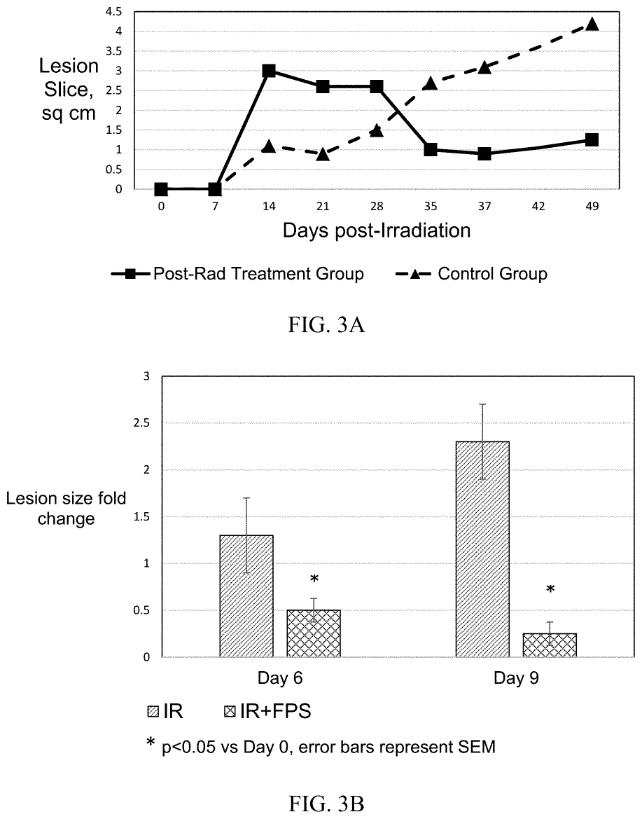

2—FPS stimulates self-repair of multiple-tissues promoting stem cells mobilization. The administration of FPS was found to increase the count of White Blood Cells (WBC) in rats when administered either as a mitigator or as a therapeutic indicating that FPS also offers protection against neutropenia (Not shown). The subgroups of white cells (lymphocytes, monocytes, granulocytes) all followed the pattern of the overall WBC, suggesting that the precursor cells of the WBCs have been affected positively stimulated by the administration of FPS. Additional studies demonstrated that FPS promotes stem cells mobilization towards the skin lesion. Needle or punch biopsies of the skin injuries of irradiated control rats and irradiated rats treated with FPS (for 2 days after injury appearance) were obtained after Day 9 and analyzed by immunological staining, using DAPI and anti-CD34 antibody. The FPS treated group showed a significantly higher red fluorescence intensity near the injury site compared to the control group, as shown in (FIG. 3b, p<0.05). This indicates a higher concentration of CD34+ cells around the ulcer in the FPS treated group, supporting the hypothesis of FAS mechanism of action.

It can be appreciated that DAPI can be, but not limited to 4′,6-diamidino-2-phenylindole, or any fluorescent stain that binds strongly to adenine-thymine-rich regions in DNA. It is used extensively in fluorescence microscopy. As DAPI can pass through an intact cell membrane, it can be used to stain both live and fixed cells, though it passes through the membrane less efficiently in live cells and therefore provides a marker for membrane viability.

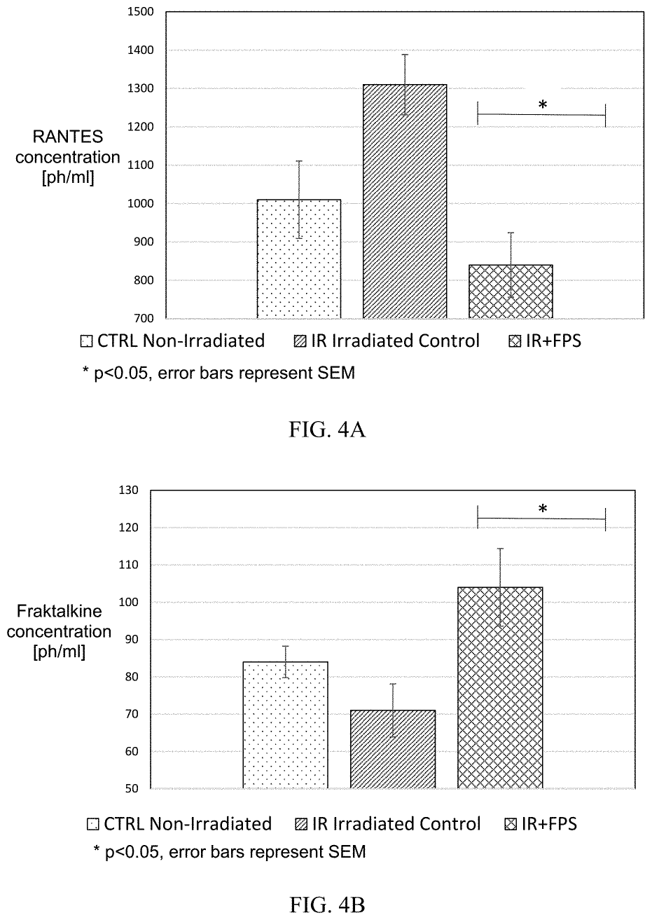

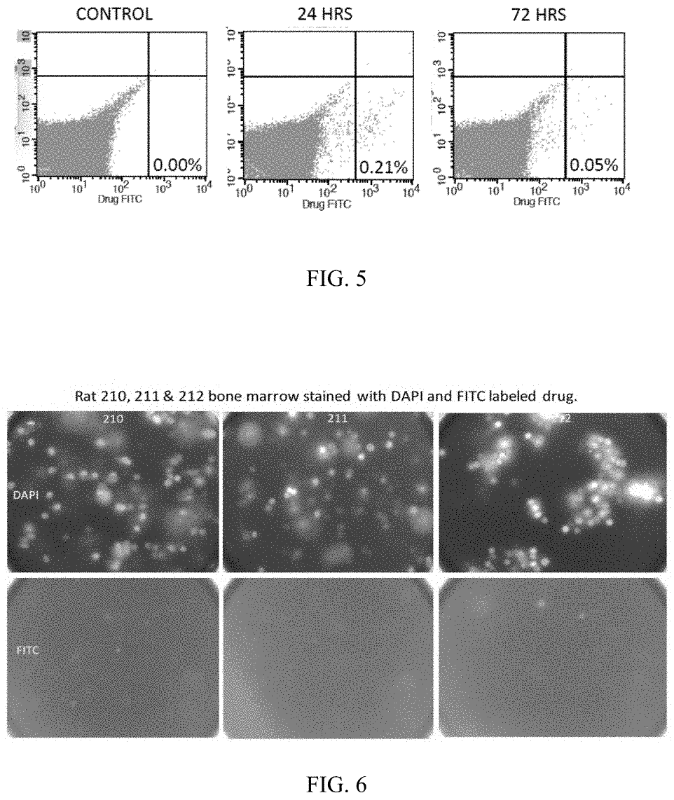

3—FPS reduce the inflammatory state of radiation wounds by regulating the cytokines profile. The inflammatory and proliferative phases of wound healing in RSI are disrupted by the early effects of radiation exposure, resulting in an impaired wound healing process. Remarkably, after FPS treatment, several cytokines were significantly altered by FPS treatment. In particular, FPS was shown to ameliorate the inflammatory response by reducing the amount of RANTES secreted by endothelial cells (FIG. 4A, p<0.05) and to promote the stem cell development by increasing the production of fractalkine (FKN) (FOG. 4B, p<0.05). RANTES is a chemokine expressed by hematopoietic cells that plays an important role in homing and migration of T cells during acute infections⁴¹, whereas fractalkine promotes chemotaxis and adhesion of leukocytes and supports the survival of multiple cell types during homeostasis and inflammation⁴¹. The expression of monocyte chemoattractant protein-1 (MCP-1, one of the key chemokines that regulates migration and infiltration of monocytes⁴¹) was down regulated in the irradiated group and enhanced in FAS treated group which showed additional modulation of the immune response (Control=789±129 pg/ml, IR=628±89 pg/ml, IR+FPS=1090±152 pg/ml).

### Rationale for the Project.

This project or present technology includes the study of the impact of two different kinds of trauma to the same patient, namely that of a deep mechanical wound (from a surgical instrument) and a moderate dose of irradiation (as used in standard radiation therapy.) Whether there is a difference if irradiation occurs before surgery (Aim 1), or whether surgery occurs before irradiation (Aim 2) will be evaluated. If there is a difference, it would confirm the complexity of polytrauma treatment, because which trauma (out of the several traumas) happens first may make a difference to the treatment modality. In subsequent studies, we may include the effect of adding a chemotherapy to the animal, to see if the presence of a toxin (the chemotherapy) will further complicate the treatment approach.

Previous studies demonstrated the efficacy of FPS as MCM for the treatment of radiation-skin injuries induced by high doses of radiations and showed how FPS target key pathways in the wound healing process. These results support a possible use of FPS in the management of wound healing in radiotherapy (RT) patients after exposure to low-medium intensity radiations. While these patients might experience mild RSI, the disruption of the molecular pathways responsible for tissue healing represents a challenge for the management of surgical wounds. The goal of this project is to perform the first preclinical assessment of FPS efficacy in supporting healing of surgical wounds altered by radiotherapy. If confirmed, FPS could accelerate healing of surgical wounds altered by the radiotherapy improving outcomes of surgical interventions as well as shortening the time window between RT and surgery with positive impact on cancer treatment.

A murine orthotopic metastatic breast cancer model⁴² will be used to mimic human breast cancer progression and treatment courses. Radiation therapy is an essential component of multimodal treatment of breast cancer and it has been shown to improve survival rates and decreases local recurrence. However, the deleterious effects post-mastectomy RT on wound healing challenges breast reconstruction, leading to delays for up to a year after radiation before performing breast reconstruction. On the other end, neoadjuvant (preoperative) RT has been shown to reduce the risk of secondary tumors in early-stage breast cancer patients' as well as downsizing locally advanced breast cance⁴⁴, but increases risk of surgical complications.

In this study, some or all embodiments of the present technology will validate the efficacy of FPS in promoting wound healing in both the scenarios. In parallel, some or all embodiments of the present technology will assess for the first time the mode of action of FPS in a tumor-bearing preclinical model. All previous studies on FPS have been performed on naïve preclinical models. However, pathological states including cancer can strongly influence sensitivity to drugs and biological agents⁴⁵. Different sensitivity due to the tumor environment may translate to changes in the efficiency of FPS observed on the stem cells mobilization, modulation of cytokines profile, augmentation of hemostatic function. Activities will be organized in 2 aims organized according to the Gantt chart.

Aim 1: Demonstrate the Ability of FPS to Promote Surgical Wound Healing after Neoadjuvant Radiotherapy.

Rationale. Some or all embodiments of the present technology will first validate the positive effect of FPS in the healing of surgical wound in the presence of pre-operative radiotherapy regimens. Irradiation is known to kill stem cells more than mature cells. Previous results indicated that administration of FPS before radiation could affect the stem cells mobilized by FPS. To avoid this risk even in low dose irradiation, FPS in this project will be given always following radiation and not before the irradiation event.

### Task 1.1A Model Generation & Treatment.

A total of 50 Orthotopic Metastatic Breast Cancer mice will be generated. To achieve a higher tumorigenesis rate with minimal tumor size variability compared to other breast cancer inoculation techniques, mouse mammary adenocarcinoma cells expressing luciferase (4T1-luc2 cells) will be inoculated at the #2 mammary fat pad under direct vision through a median anterior chest wall incision. This technique produces a high tumorigenesis rate with less variability in tumor size and shape compared to subcutaneous inoculation, as the subcutaneous microenvironment is quite different from the mammary gland microenvironment⁴⁶. Primary tumor burden will be measured daily by bioluminescence measurement. At 7 days after injection a visible primary tumor is expected to be detected. Mice will be randomly assigned to 5 study groups as described in Table 4.

TABLE 4

| | Treat- | | | Day x | Day of |
|---|---|---|---|---|---|
| Study | ment | Day 8: | Day 9: | (tumor | Sacri- |
| group | Summary | RT | Dosing | size 150%) | fice |
| #1 | Mock | No | No | Nothing | Tumor |
| | | | | | size 200% |
| #2 | RT + Saline | Yes | Saline | Nothing | Tumor |
| | | | | | size 200% |
| #3 | RT + FPS | Yes | FPS | Nothing | Tumor |
| | | | | | size 200% |
| #4 | RT + | Yes | Saline | Surgery | #5 wound |
| | Saline + | | | | >10% better than |
| | Surgery | | | | #4 animals |
| #5 | RT + | Yes | FPS | Surgery | #5 wound |
| | FPS + | | | | >10% better than |
| | Surgery | | | | #4 animals |

Except for the control group 1, all the other mice will be irradiated at Day 8 after cell injection with a neoadjuvant human dose-equivalent radiation regimen of 5 fractions of 5.6 Gy/fraction (total 28 Gy) as previously established (typical human dose 50-60 Gy administered as 2 Gy fractions over 5 to 6 weeks). Fractions will be administered using a Philips RT250 orthovoltage unit (250 kV X-rays, 15 mA; Kimtron Medical, Oxford, Conn.). Radiation will be delivered in a radiation chamber with the mice covered with a protective lead shield with only the lower abdominal area exposed. One day after radiation mice will be IV dosed with 1 ml/kg saline solution (groups 2 and 4) or FPS solution, corresponding to 8 mg/kg FAS nanospheres (groups 3 and 5).

Group 4 and 5 will be additionally subjected to a radical mastectomy at Day x after irradiation (y after cell injection) to remove tumor. Day x will be determined by the continued growth of the tumor mass, being the day when any of the mice in the same treatment group showed that the tumor size is 150% that of the size on Day 7 (increased by another 50%); Day y being Day x+8 days. The orthotopic breast tumor will be resected with the surrounding tissues and axillary lymph nodes as previously described⁴². Briefly, mice will be anesthetized with inhaled isoflurane and inject buprenorphine before performing a 5 mm skin incision 2 mm to the left from the surgical scar made at the initial cancer cell inoculation. The incision will be extended toward the root of the forelimb to remove the tumor, the skin including the surgical scar, and the lesion in contact with the tumor, as well as the axillary lymph node basin in which most of the time no visible lymph node exists at the time of the mastectomy.

Regarding the day of sacrifice, all the mice in each of the Group 1, 2, 3 will be sacrificed when any one of the animals in the given treatment Group showed tumor size larger than 200% that of the size in Day 7. The tumor sizes in animals in Group 4, 5 are expected to be minimal because of the mastectomy. Therefore, the day of sacrifice will be the day when the surgical wound in Group 5 showed improvement by at least 10% than those in Group 4, using any non-invasion evaluation methods, e.g. blood flow beneath the surgical wound.

Task 1.1.B Surgery first model:

Alternatively, the impact of having surgery first (followed by radiation therapy, or not) will be assessed, as shown in Table 5, which will be described in further details in Aim 2 below.

TABLE 5

| | | |
|---|---|---|
| Study group | Treatment | Analysis |

| | |
|---|---|
| #1B | Mock |
| #2B | Surgery + saline |
| #3B | Surgery + FPS |
| #4B | Surgery + radiation + saline |
| #5B | Surgery + radiation + FPS |

It should be noted that in Group #5B, the FPS is administered one day after the dose of irradiation, instead of the order “surgery+FPS+radiation.” The reason is because stem cells are sensitive to the effects of radiation. If the order is “Surgery+FPS+radiation” then the stem cells mobilized by the FPS will be killed by the radiation dose, leading to less pronounced benefit from the administration of FPS. Because of this, the order of administration in Group #4B is also “Surgery+radiation+saline” instead of “Surgery+saline+radiation.”

### Task 1.2 Assessment of Wound Healing Process.

FPS efficacy will be assessed by measuring daily the progress (healing) of the wound lesion extension in Groups 4 and 5 as well as blood perfusion will be monitored through tissue blood flow and temperature monitoring.

On a day when any of the mice in any treatment group showed a tumor size 200% that of the size on Day 7 (Group 1, 2, and 3) all the mice in the groups will be sacrificed. On a day when any of the mice in Group 5 showed improvements in the wound healing of more than 10% better than those mice in Group 4 (by any noninvasive methods of assessment, e.g. by blood flow under the skin), these mice in Group 4 and 5 will be sacrificed: to obtain blood for the validation of the biomarkers and any histological study of the healing process.

Blood samples will be collected from a vein on the day of sacrifice in all the mice to count red blood cell counts, platelets and white blood cells. Proinflammatory cytokines will be measured to assess any possible influence of FPS administration on their overexpression after RT (Group 2 and 3) as well as after RT and surgery (group 4 and 5). In particular, growth factor beta (TGFβ), vascular endothelial growth factor (VEGF), interferon-γ (IFN-γ) and proinflammatory cytokines interleukin-1 and interleukin-6 will be measured by ELISA assays and compared with levels measured in control group 1.

Biomarkers which may be helpful in the prediction of the effective dose of FPS in healing more severe injuries such as would be found in polytrauma cases or under the Animal Rule will also be studied. The biomarkers may include and not limited to the following: mature cells, progenitor cells, stem cells, cell-communicating signals such as cytokines, cells with altered oxidative/reductive states, or intra-bone-marrow cells.

### Validation of Mode of Action.

The presence of the surgical wound is expected to send a stronger signal that the area “needs help” to heal therefore a stronger signal to mobilize the stem cells (or other intermediate cells) to the wound is expected. The mobilization of stem cells will be assessed following the protocol described by Cotola, A. et al. (2018) in the different study groups. For the flow cytometry count of circulating stem cells, anti-CD34 antibody will be used to label and visualize the presence and concentrations of progenitor cells amidst the regenerating process. The concentration of CD34+ cells will be measured by flow cytometery, and the number of CD34+ cells in peripheral blood (PB) will be calculated as the count of absolute WBC times the percentage (%) of gated CD34+ positive cells, and expressed as absolute number of cells per 1 μl PB. Needle or punch biopsy of the wound site will be also performed on the day of sacrifice and assessed by immunological staining, using anti-CD34 antibody or any other appropriate cell surface markers. In addition to PB, the number of CD34+ cells will be counted per unit area in random biopsies of tissues obtained from around the wound, fixed and stained on microscopy slides. We expect a larger concentration of CD34+ cells around the wound in the FAS-treated group compared to the control group treated with normal saline.

### Task 1.3. Safety Assessment.

Even if toxicological studies have already confirmed the safety of FPS, body weight will be assessed every day until day of sacrifice to confirm any indication of potential toxicity in a cancer environment: an average weight loss in FPS-treated groups more than 10% that of the normal saline-treated group without other explanations will be considered an adverse effect. Even with a relatively small size of sampling, the FAS group is expected to show statistically (p<0.05) improvement in reduction of the area of the skin lesion with respect to the control group. We will compare the average maximal wound area between control and treated group (p-value <0.05). A one-way ANOVA test will be used to evaluate if there is any statistically significant difference among different FPS treatment groups (p-value <0.05). Finally, a post-hoc analysis based on two-sample t-tests will be also carried out. A power analysis performed based on previous studies with FPS and RSI showed that a group size of 10 animals is needed to reach statistically significant differences in control and treated animals regarding the quantification of biological markers associated with wound healing (power 0.8, alpha 0.05, double-sided analysis). Based on the experimental model selected, female only mice will be used.

### Milestone #1. FAS Works as Radiotherapy Mitigator in a Preclinical Cancer Model.

Expected Outcomes: Vascularization improvement of Markers changes of Wound healing 25% faster than control.

### Potential Pitfalls and Anticipated Alternative Strategy.

It is important to consider that the cancer microenvironment and surgery can affect the stress/immune response. To take care of these potential confounding factors, additional controls where FPS only will be injected into mice injected with tumor cells but no surgery, nor radiation, will be added. The expectation is that tumor cells in mice treated with saline only (control) grows at the same rate as tumor cells in mice treated with FPS only.

### Aim 2. Demonstrate the Ability of FPS to Promote Wound Healing Altered by Post-Mastectomy Radiotherapy.

Rationale. Some or all embodiments of the present technology will assess the use of FPS in the scenario of RT given after surgical treatment. As model of surgical intervention, the surgeon will perform a radical mastectomy procedure in the murine Orthotopic Metastatic Breast Cancer model previously described. While FPS has been shown to promote healing of RSI, its mode of action could in theory support healing of any wound independently of radiation. Therefore, in this aim the ability of FPS to accelerate and improve surgical wound in the absence of RT will be also assessed. If this ability is confirmed, new exciting indications could be targeted for FPS.

### Task 2.1 Model Generation & Treatment

A total of 50 Orthotopic Metastatic Breast Cancer mice will be generated as described in Aim 1 and radical mastectomy will be performed at Day 8 after cell inoculation to remove tumor. Seven days after surgery or earlier, sutures will be removed under anesthesia and treatment will be performed. Mice will be randomly assigned to 5 groups (10 mice per group) as indicated in Table 6.

TABLE 6

| | Treat- | | | | Day of |
|---|---|---|---|---|---|
| Study | ment | Day 7: | Day 8: | Day 9: | Sacri- |
| group | Summary | Surgery | RT | Dosing | fice |
| #0 | Mock | No | No | No | Tumor |
| | | | | | size 200% |
| #1 | Surgery + | Yes | No | Saline | #3 wound |
| | Saline | | | | >10% better than |
| | | | | | #2 animals |
| #2 | Surgery + | Yes | No | FPS | #3 wound |
| | FPS | | | | >10% better than |
| | | | | | #2 animals |
| #3 | Surgery + | Yes | Yes | Saline | #5 wound |
| | RT + Saline | | | | >10% better than |
| | | | | | #4 animals |
| #4 | Surgery + | Yes | Yes | FPS | #5 wound |
| | RT + FPS | | | | >10% better than |
| | | | | | #4 animals |

Group 1 mice will be allowed to grow tumor size with no interference. Mice in Group 2, 3, 4, 5 will all have mastectomy on day 7 so as to keep the administration of RT (for Group 4, 5 mice) on the same day as in Aim 1 (which is Day 8). To administer RT within one day of surgery may be considered aggressive, but it will provide the best scenario to show if FPS administration will have a positive effect.

Group 2 and 3 will be dosed with saline solution or 8 mg/kg FPS, respectively on Day 9, the same day as done in Aim 1. While Group 4 and 5 will first receive a post-mastectomy human-dose-equivalent radiation regimen on day 8, of 5 fractions of 5.6 Gy/fraction (total 28 Gy) as previously established⁴⁷ (typical human dose 50-60 Gy administered as 2 Gy fractions⁴⁸ over 5 to 6 weeks). Saline and FPS will be then administered in these two groups, respectively, on day 9.

### Task 2.2 Assessment of Wound Healing Process.

Group 2 and 3 will be compared to validate the ability of FPS to improve wound healing in the absence of RT. Similarly, Group 5 will be compared with Group 4, to identify the ability of FPS to improve wound healing after the administration of RT which was given after mastectomy. The assessments will be made by measuring daily the progress (healing) of the wound lesion as well as blood perfusion will be monitored through tissue blood flow and temperature monitoring.

On a day that the tumor size in Group 1 mice showed a size larger than 200%, these mice will be sacrifice. For mice in Group 2 and 3, they will be sacrificed when any of the mice in Group 3 showed improvements of the wound lesions better than those in Group 2 by larger than 10% (using any noninvasive method). Similarly, for mice in Group 4 and 5, they will be sacrificed when any of the mice in Group 5 showed improvements of the wound lesions better than those in Group 4 by larger than 10%.

Blood samples will be collected on the day of sacrifice for all groups. The samples will be used to count red blood cell counts, platelets and white blood cells. Proinflammatory cytokines will be measured to assess any possible influence of FPS administration on their overexpression after RT (Group 2 and 3) as well as after RT and surgery (group 4 and 5). In particular, growth factor beta (TGFβ), vascular endothelial growth factor (VEGF), interferon-γ (IFN-γ) and proinflammatory cytokines interleukin-1 and interleukin-6 will be measured by ELISA assays and compared with levels measured in control group 1.

Biomarkers which may be helpful in the prediction of the effective dose of FPS in healing more severe injuries such as would be found in polytrauma cases or under the Animal Rule will also be studied. The biomarkers may include and not limited to the following: mature cells, progenitor cells, stem cells, cell-communicating signals such as cytokines, cells with altered oxidative/reductive states, or intra-bone-marrow cells.

### Validation of Mode of Action.

The mobilization of stem cells will be assessed following the protocol described by Cotola, A. et al. (2018) in the different study groups. For the flow cytometry count of circulating stem cells, anti-CD34 antibody will be used to label and visualize the presence and concentrations of progenitor cells amidst the regenerating process. The concentration of CD34+ cells will be measured by flow cytometry, and the number of CD34+ cells in peripheral blood (PB) will be calculated as the count of absolute WBC times the percentage (%) of gated CD34+ positive cells, and expressed as absolute number of cells per 1 μl PB. Needle or punch biopsy of the wound site will be also obtained on the day of sacrifice for all mice and assessed by immunological staining, using anti-CD34 antibody and any other appropriate cell surface markers. In addition to PB, the number of CD34+ cells will be counted per unit area in random biopsies of tissues obtained from around the wound, fixed and stained on microscopy slides. We expect a larger concentration of CD34+ cells around the wound in the FAS-treated group compared to the control group treated with normal saline.

### Task 1.3. Safety Assessment.

Body weight will be assessed every day to confirm any indication of potential toxicity in a cancer environment: an average weight loss in FPS-treated groups more than 10% that of the normal saline-treated group without other explanations will be considered an adverse effect.

### Milestone #2. FAS Promotes Better Outcomes of Post-Radiotherapy Surgery.

### Expected Outcomes:

Wound healing improved of at least 25%

Vascularization improvement of at least 15%

Biomarkers changes of at least 20%

### Other Applications of FAS in Polytrauma

Although the above section provides details of how FAS can be studied in one situation (that of a combination of surgical wound plus radiation) other conditions of polytrauma are expected to benefit from the timely administration of FAS, including the prophylactic, mitigative and therapeutic approaches. The various conditions of polytrauma can include non-skin-breaking conditions (e.g. blunt trauma where no obvious skin is broken) and hard tissues (bone) damage. In the situation of a combat injury, it can be expected that burn injuries will be added to blunt trauma and bone fractures, because practically all projectiles (missile fragments, shell casings, explosives) are hot. The benefit of FAS administration will be studied in any of the above-mentioned polytrauma conditions.

The Source of Regenerative Cells Capable of Healing the Various Injuries Caused by More than One Kind of Trauma.

Introduction: Stem cells appear to be good candidates which can travel via the blood stream to a wound and regenerate the various tissues at the wound depending on where a given stem cell adheres to (e.g. new bone is regenerated at a site where bone had been injured and new bone is needed for recovery.) Conventionally, stem cells are considered to have all originated from the bone marrow. However, studies in the regeneration of blood vessels have shown that while “ultimately” all stem cells might have originated from the bone marrow at the beginning of their lineage, there are intermediate cell populations which are important to the regeneration of blood vessels. We expect the same to be true eventually for the different types of tissues or organs that need replacement of damaged cells in that particular tissue or organ.

An example of the intermediate cell population providing regenerative cells for blood vessel regeneration can be found in Curr Opin Hematol. 2014 May; 21(3): 224-228. doi:10.1097/MOH.0000000000000041. The title is “Endothelial Progenitor Cells and Vascular Repair” with the first author Min Zhang. The abstract stated: Purpose of review—Identify recent advances in the field of vascular repair by regenerative endothelial cells (ECs) and endothelial progenitor cells (EPCs). It discloses its recent findings—A growing number of studies indicate that bone marrow derived circulating EPCs do not engraft into blood vessels, but that such circulating cells may regulate vascular repair via paracrine mechanisms. Novel modes of paracrine regulation are being uncovered, such as the release of EC-derived microparticles or microvesicles which contain microRNAs that can promote vascular repair. Instead of circulating cells, tissue resident ECs or EPCs may primarily drive the restoration of vascular function after endothelial injury. In addition to the generation of ECs/EPCs from pluripotent stem cells, direct reprogramming of fibroblasts to ECs/EPCs is becoming an important source of regenerative vascular cells.

The authors summarize the finding as follows: Ongoing efforts to understand the mechanisms that regulate vascular repair by resident regenerative vascular cells as well as their generation from fibroblasts and pluripotent stem cells will form the basis of future regenerative therapies.

### Considerations for Healing Mechanisms Under Conditions of Polytrauma or Severely High Doses of Irradiation.

While Zhang et al. discussed the contribution of resident endothelial cells (i.e. endothelial cells that are still living in the endothelium after the injury) and potentially other circulating cells in the blood toward the rebuilding of the endothelium, one must be mindful that in the case of polytrauma, the mechanism of regeneration may be very different. One obvious situation is the aftermath of a severely high dose of irradiation which overwhelms the ability of the resident endothelial cells to regenerate new cells, or the mass (or volume) of cells having been destroyed simply cannot be replaced by barely alive cells still present in the neighborhood. In addition, the bone marrow which acts as the ultimate source of all stem cells may have also been so adversely affected that the “normal” mechanisms of regeneration may not work or may have been severely delayed.

Considerations for where to Look for Regenerative Cells that Will Indicate that the Wound would Eventually be Healed.

CD34 is a cell surface marker generally correlated with stem cells. FIGS. 3A and 3B shows that the concentration of CD34+ cells are more abundant at the biopsy site of the FAS-treated group (FIG. 3B) than at the biopsy site of the normal-saline-treated group (FIG. 3A). The data showed that after the “combination” of irradiation injury plus FAS administration, there are more CD34+ cells which had arrived at the wound site than in the control group (which has been irradiated but only received normal saline.)

To fully understand the complex situation of wound healing, one needs to remember there are at least 3 sites to examine. (a) The “site of origin” of the stem (regenerative) cells. One can assume that the origin site is mainly the bone marrow but it could be another site. (b) The “transit” site, i.e. blood (because the stem cells have to move from its site of origin to the injury site.) This is often studied because blood can be sampled from patients without adding further major injury, such as attempts to study the bone marrow of an injured person or taking biopsy at the wound site before it is healed. However, there may be only a few days when the transit of stem cells in the blood will peak—one can easily miss collecting blood during this peak period. (c) The wound site, which is the “destination site” of the stem cells. However, studies at the wound site can be difficult because the “wound” may not always be visible. In the case of irradiation, the “wound” can be millions of leaky endothelial sites inside the body and too small to be visible by any instruments. Also, stem cells are supposed to be differentiating to become mature cells at the (larger and more visible) wound site, e.g. muscles and nerves, which would have shed the markers designated for stem cells, e.g. losing the CD34 marker all together while the cells have become CD34 negative cells.

The issue of differentiation is also a challenge. One could expect that the stem cells would become different kinds of (more mature) cells sometime after their arrival at the injury site. However, this does not mean the “young stem cells” cannot go through intermediate stages of differentiations before they become functional and mature cells at the destination site. That is why histologists and hematologists have constructed different “cell lineages” e.g. for the white blood cells and the various subsets of white blood cells. In terms of stem cells “signaled to” or “called” to arrive at the injury site, they may have different pathways of differentiation depending on the severity of injury and the location of the damage.

The detection of these CD34+ cells (as shown in FIGS. 3A and 3B) even after they have left their site of origin and have arrived at the target site (healing wound site after the irradiation injury) is only looking at one aspect of the healing process. The fact that these cells are still stainable as CD34-positive at the wound site of the FAS-treated animals on Day 9 after the appearance of the skin lesion means these cells have not yet fully differentiated (or regenerated) into muscles, or nerve cells, or blood vessel cells, which would have shed the CD34+ marker by then. Since the histology study is aimed at detecting CD34-positive cells, the process would have missed many of the previously CD34+ cells that have already differentiated into other (mature) cell types. Therefore, if one focused on the ratio of CD34+ cells in the FAS-treated group vs the control, one would have underestimated the speed with which the tissues are healing, because the more CD34+ cells have differentiated via the healing process into mature cells, the fewer CD34+ cells would have been left in the wound to be detected by the histological staining process. The fact that the FAS-treated group is really healing is validated by the observation that the visible skin lesion is almost ten-fold smaller on Day 9 (after the appearance of the lesion) in the FAS-treated group vs the control group. In other words, even if the histological staining fails to show an abundance of CD34+ cells at the wound in the FAS-treated group, it does not necessarily mean that there is no healing at the macroscopic level: the gold standard is still how well the wound (if visible) is healing; the presence of CD34+ cells (more so in the drug-treated group over the control group) is really only a biomarker in case it is hard to visualize the wound(s) itself.

The Role of the Bone Marrow to Produce Regenerative Cells (Stem Cells): Are Stem Cells Mobilized after Trauma, with or without the Administration of Fibrinogen-Coated Albumin Spheres (FAS); or would Stem Cells be Stimulated/Mobilized by FAS Alone Even in the Absence of Injury?

The following questions need to be asked: (a) Will the administration of FAS alone (in the absence of injury or other stimuli) induce the mobilization of stem cells? In that case, since there is no wound site, there is no site to take a biopsy to perform histological staining to visualize the concentration of CD34+ cells, to compare the drug-treated group with the control group. One would have to study the cell population within the bone marrow (or another known site of origin) to detect any increase in the increased concentration of stem cells. (b) In a situation of mild injury, one would expect to see an increase in the concentration of stem cells in either the bone marrow and/or the blood, because the wound is expected to heal eventually. However, if the injury is so severe that it will not heal (such as after a nuclear blast in war or after an industrial accident) the failure to heal can be due to an insufficient mobilization of stem cells, or it could be due to failure to cover the loss even when stem cells are maximally produced and moving toward the wound or wounds. (c) In the “combined event” that an injury has occurred and an adequate dose of FAS is administered, will there be a synergistic effect between the stimuli from the injury and the stimuli from the drug?

If FAS alone can stimulate or mobilize stem cells from their site of origin, it means FAS can open the door to “stem cell therapy” for diseases that are not necessarily caused by external sources of trauma or recognized incidents of injury. Patients with long term diseases such as Alzheimer's diseases and other degenerative diseases may benefit from the administration of FAS.

Regarding the effect of FAS alone on animals without known injury, including irradiation-induced skin damage, Mao et al. have published their findings in abstract form in Radiation Research, 2015 titled “Fibrinoplate-S for the Treatment of Radiation-induced Skin Damage.” The data showed that there are statistically higher concentrations of white blood cells and their subsets in the blood of FAS-treated rats (without irradiation) than in control rats without FAS and without irradiation. What is noteworthy is that the blood was obtained on the day of sacrifice which was 51 days post-irradiation. The data strongly suggest that if one were to study any of the following after FAS administration, (a) the site of origin, the bone marrow, (b) the site of transit (blood) before Day 51 post-irradiation, (c) the destination site, i.e. site of repair (the healing wound in the FAS group if there is a wound to be repaired)—one would expect to find increased concentration of progenitor cells to the white blood cell line.

Therefore, it is intended to initiate the following study titled “The effect of FAS on concentration of CD34+ cells in the mouse bone marrow.”

There will be 5 groups of mice, Group A is control with normal saline infusion; Group B, C, D, E will be mice administered FAS intravenously and sacrificed on Day 1, 5, 10, 14, respectively. Groups of 4 CD1 female mice will be treated with the drug (FPS) by IV administration via the jugular vein catheter at 40 mg/kg on Day 0. Group A of 4 matched control mice will be treated with vehicle (normal saline, at 5 mL per kg) alone. Prior to sacrifice, blood will be collected and analyzed for CBCs. At the time of sacrifice, bone marrow will be harvested from all mice, the red blood cells from the marrow samples are lysed and the remaining cells washed, counted a dual-stained for CD34FITC and APC lineage cocktail, to identify different subpopulations of CD34+ cells. According to the supplier, the APC Mouse Lineage Antibody Cocktail has been designed to react with cells from the major hematopoietic lineages, such as T lymphocytes, B lymphocytes, monocytes/macrophages, NK cells, erythrocytes, and granulocytes. This pre-diluted Cocktail of five APC-conjugated antibodies is designed for the flow cytometric identification of hematopoietic progenitors in mouse bone marrow. Components include clone 145-2C11, which recognizes Mouse CD3e; M1/70, which recognizes CD11b; RA3-6B2, which recognizes CD45R/B220; TER-119, which recognizes Ly-76, mouse erythroid cells; and RB6-8C5, which recognizes Ly-6G and Ly-6C. Please see example of staining below. Cells will be analyzed by flow cytometry on a FACSCalibur flow cytometer.

The results will show that the hematopoietic lineages within the bone marrow do show increase with time after the administration of this dose of FAS. It should be noted that the effective dose of FAS for the healing of a skin injury caused by a locally administered dose of irradiation at the skin of the hind leg, at 25 Gy, is only 8 mg/kg in the rat. The FAS is administered in two doses, which is effective regardless of whether the FAS is administered on Day −2, −1 (prophylaxis); on Day +1, +2 (mitigation) post-irradiation, or at Day 1 and 2 post-lesion appearance. The data suggests that young hematopoietic cells in the bone marrow will be good markers to predict the effectiveness of FAS should a situation arises where it is appropriate to harvest bone marrow cells in potentially endangered patients who is expected to encounter an injury that can be healed by FAS administration.

### Experimental Data

### Experiment 1: Accumulation of Fps in Rat Bone Marrow Cells

### 1.1. Purpose:

The purpose of this study is to determine whether a fluorescein-labeled FAS (Fibrinogen-coated albumin spheres, the active ingredient or drug substance) can be taken up after one dose of intravenously administration, by bone marrow cells in a Sprague-Dawley rat model, using flow cytometry and fluorescence microscopy/imaging techniques.

TABLE 7
Experimental Design

| | Number of | Animal |
|---|---|---|
| Treatment | rats per group | id # |
| Control (saline 4 mL per kg) | 1 | 211 |
| FITC-FAS Treatment: bone | 1 | 210 |
| marrow harvested 24 hrs later | | |
| FITC-FAS Treatment: bone | 1 | 212 |
| marrow harvested 72 hrs later | | |

### 2. Materials and Methods

The FITC-FAS was prepared by aseptic techniques using FITC (fluorescein isocyanate) to label the spheres in the original drug product called Fibrinoplate-S (FPS, containing 8 mg of spheres suspended in a supernatant containing sorbitol and sodium caprylate.) The FITC-FAS (to be called “drug” here) was suspended in sterile normal saline. It passes the release parameters normally used for release of the drug product. The final concentration of FITC-FAS in the suspension is at least 6 mg of spheres per mL (in saline alone.) The “specific activity of FITC on the FITC-FAS spheres” is at least 4 ug FITC per mg sphere. The amount of “free FITC” is less than 5% of the total amount of FITC in the suspension.

Three Sprague Dawley Female Rats instrumented with double jugular catheters were treated via slow IV bolus (1-2 min) with the FITC-FPS or vehicle alone (normal saline) at the dose of 32 mg/kg. This is a dose about 4 times that of the effective dose (8 mg per kg) used in a rat model for healing of radiation-induced skin injury. Bone marrow was harvested at two different time-points after drug treatment (24 or 72 hours post FITC-FAS dosing.) Bone marrow was collected and cells were counted and divided into two portions. One half was used to generate cell smears for microscopic examination and the other was analyzed by flow cytometry.

### 2.1. Cell Smears.

Cells were washed, and smears were prepared on glass slides. Smears were mounted in Prolong Slow-fade Gold mountant containing DAPI (Fisher Scientific) and examined using a Zeiss Axioskop fluorescence microscope. Cells were observed for green fluorescence indicating FITC-FAS uptake or binding, and the intracellular location of the drug. DAPI stains all nuclei and gives an indication of the total number of cells per field. Photomicrographs showing either DAPI or FITC staining, as well as overlays, were generated.

### 2.2. Flow Cytometry.

Red blood cells were lysed using BD Cell-lysing buffer (Becton Dickinson) according to the manufacturer's directions. Cells were washed and filtered through 70 uM cell strainers prior to flow cytometric analysis using a BD FACSCalibur flow cytometer to determine the frequency of fluorescent drug positive cells. Data was analyzed using CellQuest Software (Becton Dickinson).

### 3. Results

TABLE 8
Bone marrow cell recoveries from rat limb bones

| | Animal | # bone marrow |
|---|---|---|
| Treatment | id # | cells harvested |
| Control | 211 | 73.4 × 10⁶ |
| FITC-FAS Treatment: | 210 | 90.4 × 10⁶ |
| harvest 24 hrs | | |
| FITC-FAS Treatment: | 212 | 45.6 × 10⁶ |
| harvest 72 hrs | | |

### 3.1. Flow Cytometry.

Cells were analyzed on a Becton Dickinson FACSCalibur flow cytometer using 488 nm excitation (Argon laser) and 530 nm emission (bandpass filter). Cells were gated on forward and side scatter parameters expected to include nucleated cells and to exclude any residual red blood cells, platelets, debris and or unbound drug (although these should have been removed by the wash steps used). 50,000 gated events per sample were collected. Cells were analyzed using Cell Quest Pro software. The percentage of positive cells is shown in Table 9 below. Only a small percentage of cells were FITC positive, consistent with the fluorescence microscopy observations. The percentage of positive cells peaked at 24 hrs and was reduced by 72 hrs after drug administration.

TABLE 9
FITC-positive cells (% of all cells)

| | Animal | % FITC |
|---|---|---|
| Treatment | id # | positive cells |
| Control | 211 | 0.00 |
| FITC-FAS Treatment: | 210 | 0.21 |
| harvest 24 hrs | | |
| FITC-FAS Treatment: | 212 | 0.05 |
| harvest 72 hrs | | |

### 3.2. Cell Smears

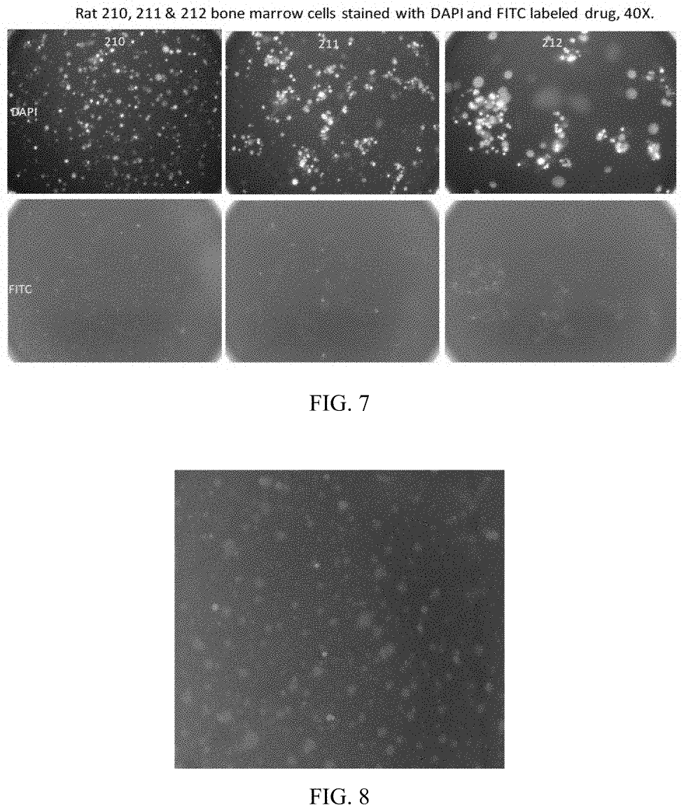

Observation using a fluorescence microscope using filters for DAPI showed numerous cells identified by the nuclei stained with DAPI, as shown in FIGS. 6-7. However, using filters for FITC revealed that the frequency of cells that had taken up the drug was very low at both time points after treatment.

### 4. Discussion

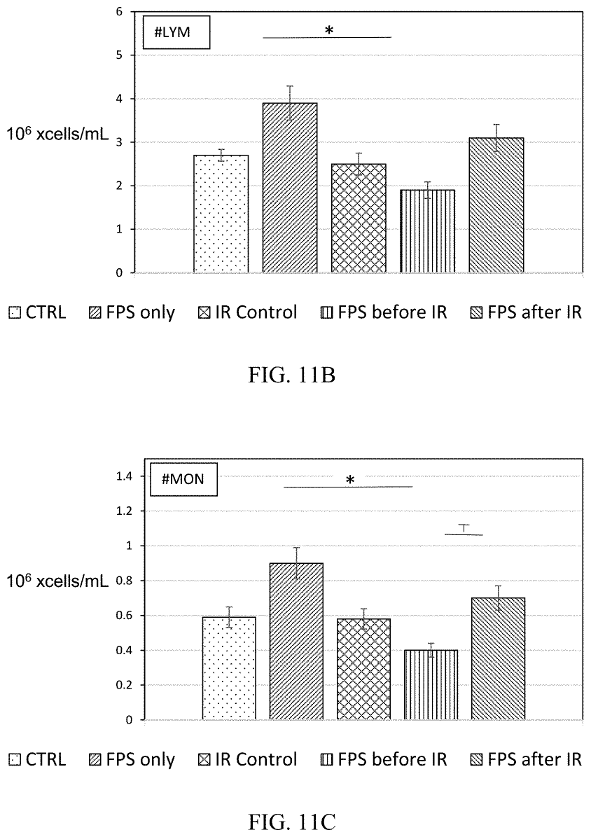

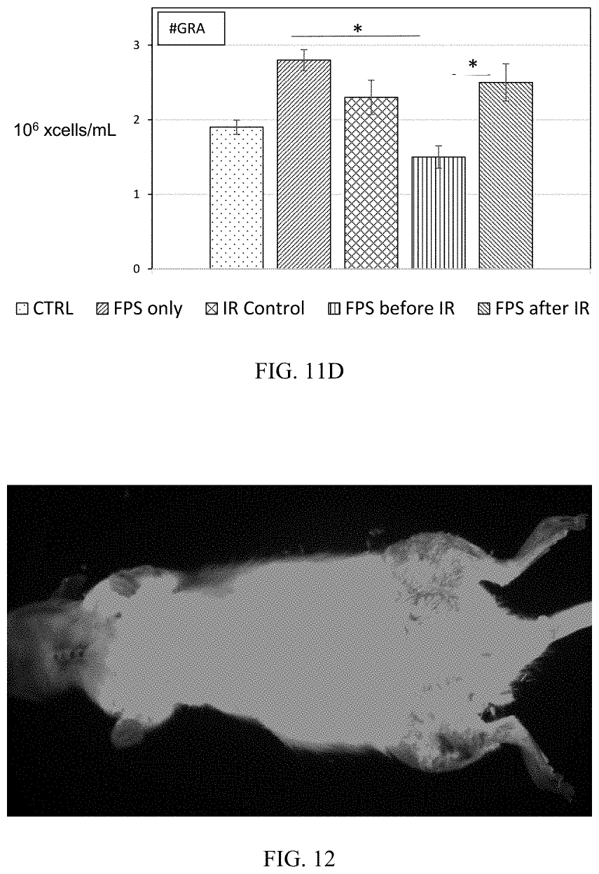

FIGS. 12-14 are whole body images of the control rat #211, the drug treated rat #210 and the drug treated rat #212. FIG. 11 is a fluorescence microscope image of a control rat with the hair completely obstruct the fluorescent image and the legs were shaved in the attempt to distinguish between the autofluorescence and possible drug fluorescence.

FIG. 12 is a fluorescence microscope image of a drug-treated rat #210 (24 hours after fluorescent drug administration). No fluorescence was seen in the leg containing bone marrow (besides the hair autofluorescence interference).

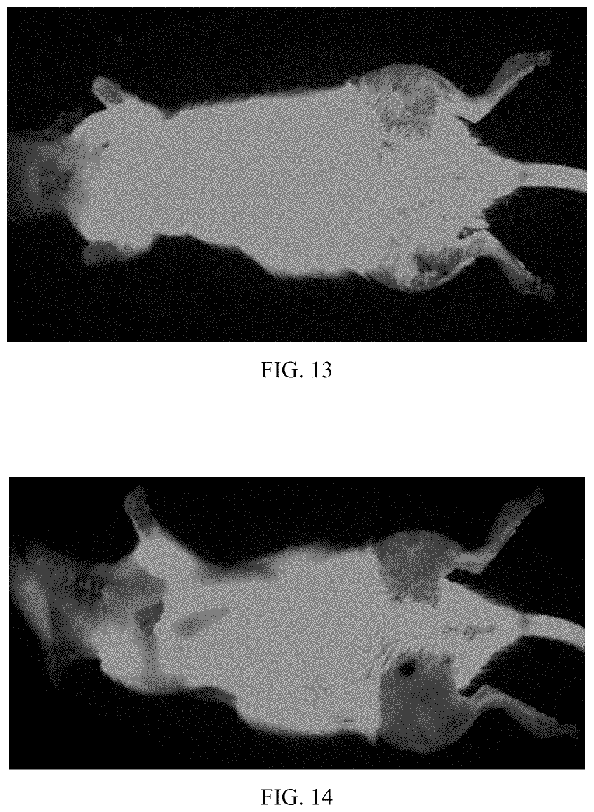

FIG. 13 is a fluorescence microscope image of a drug-treated Rat #212 (72 hours after fluorescent drug administration). No fluorescence was seen in the leg containing bone marrow (besides the hair autofluorescence interference).

The flow cytometry measurement confirmed that the FITC-FPS (drug) accumulated in the bone marrow cells of drug-treated rats. None were observed in the control rats. Although the percentage of cells with attached FITC-FAS is relatively low (approximately 0.2% of all the harvested bone marrow cells at 24 hrs after treatment) in absolute numbers, it is still around 180,000 cells (0.2% of 90 million of the cells harvested). Remarkably, FITC-fluorescence is still measurable at 72 hours after the drug administration, even though it is at this time at an even lower level (0.05%) among the harvested cells.

The overlay pictures of both DAPI with FITC fluorescence show clearly that all the FITC images are associated with DAPI-labeled cells. In other words, the FITC images are not located in locations separate from where the DAPI-cells are located. This means the FITC-FAS spheres are attached to the cells. The green fluorescence images are not likely to be due to free FITC labels ingested by the bone marrow cells. This is because (a) the amount of free FITC (not attached to the spheres) is negligible in the intravenous dose of FITC-FAS and 72 hours are plenty of time for any free FITC molecules to have left the blood compartment; (b) pilot experiments have shown that the FITC label does not detach from the FITC-FAS even after incubation with plasma for more than 24 hours. It is known that the bone marrow has a blood compartment. Since the FITC-FAS is administered via the intravenous route, the FITC-spheres must have reached the bone marrow cells via some pathway connecting the blood compartment inside the bone marrow with the marrow cell compartment.

The magnification of the Merged Images (FIG. 8) is only 40×. At this magnification, particles around 0.2 micron (the size of FITC-FAS) will not be visible under the microscope. Therefore, the bright green dots shown in FIG. 8 in animal 212 (harvested at 72 hours post-treatment) probably represent particles larger than one micron, which may be (a) aggregates of FITC-FAS formed during the processing of the cells for microscopy, even when the FITC-FAS individual spheres have already been bound to the surface of the bone marrow cells when these cells are alive; or (b) the FITC-FAS individual spheres can move around the cell surface even after their individual attachment, before the cells are smeared onto glass slides for study.

The data shown here represent the first set of data which prove that FAS (labeled or not) can enter the bone marrow after the administration of one dose of FAS (or FPS) via the intravenous route. The data lend firm support to the proposition that the administration of FAS via the intravenous route can lead to the mobilization of stem cells most of which have their site of origin in the bone marrow.

### Experiment Two

### FPS (Fibrinoplate-S) Increases the Concentration of CD34-Positive Cells in the Mouse Bone Marrow

### 2.1. Purpose of Work

The purpose of this work was to provide pilot data to show that a high dose of FPS (Fibrinoplate-S, the drug product) administered intravenously in a mouse model can stimulate the concentration of CD+ cells in the bone marrow. The drug product is the original product (unlabeled) in a suspension containing sorbitol and caprylate.

Subsequent work may use a lower concentration of FPS which is expected to be effective doses in several (and not limited to these) medical indications: (a) improvement of bleeding time in thrombocytopenic animals (including animals which are both thrombocytopenic and platelet-transfusion refractory), (b) improvement in the healing of radiation-induced skin injuries (in the rat and the mini-pig model, and potentially in the human patient); (c) accelerated healing of bone fractures in young and old rats.

### 2.2. Pilot Study to Ensure CD-34 Staining on Mouse Bone Marrow Cells Works

### 2.2.1. Material and Methods

Bone marrow was harvested from one female CD-1 mouse (Charles River), red blood cells were lysed using BD Cell-lysing buffer (Becton Dickinson) according to the manufacturer's directions. Cells were stained as follows: 1 ug of FcBlock was added to 50 ul aliquots of bone marrow cells and incubated for 10 min on ice. 1 ug of FITC anti mouse CD34 or isotype control were added and samples were incubated on ice for 20 min. Cells were washed twice and resuspended in 50 ul PBS. 3 ul of normal rat serum was added and samples were incubated for 10 min on ice. 20 ul/sample of APC lineage cocktail was added and samples were incubated for 20 min on ice. Cells were washed twice and resuspended in 200 ul PBS for FACS analysis. Cells were analyzed by flow cytometry on a FACSCalibur flow cytometer. Cells were gated on forward and side scatter parameters expected to include nucleated cells and to exclude any residual red blood cells, platelets, aggregated cells or debris. 50,000 gated events per sample were collected. Cells were analyzed using Cell Quest Pro software (Becton Dickinson).

### 2.2.2. Results

The anti-CD34 antibody stained 1.82% of CD34+ve, lineage cocktail −ve cells whereas the isotype control antibody only stained 0.19%, as shown in FIG. 9 illustrating the test anti-CD34 and anti-lineage cocktail staining of mouse bone marrow. This was within the range of expected results and therefore we proceeded to the FPS study.

### 2.3. Effect of FPS Administration on the Concentration of CD34-Positive Cells in the Bone Marrow

Table 10 shows the results from an experimental design of treatment of mice with FPA and a vehicle alone.

TABLE 10
Experimental Design

| | Time after FPS administration, | | | | |
|---|---|---|---|---|---|
| | days/mouse number per group | | | | Total # |
| Treatment | 1 | 5 | 10 | 14 | Of mice |
| FPS | 4 | 4 | 4 | 4 | 16 |
| Vehicle alone | 1 | 1 | 1 | 1 | 4 |

### 2.3.1. Materials and Methods

Fibrinoplate-S (FPS) was supplied by the manufacturer as sterile suspensions containing 8 mg of fibrinogen-coated albumin spheres (FAS) per mL, in a suspension containing up to 5% of sorbitol and 13.3 mg of sodium caprylate per gram of total protein (to prevent denaturing of proteins during the terminal pasteurization of the product in sealed bottles containing 100 mL of suspension per bottle.)

Groups of 4 CD-1 female mice (mean group weights 27.8-28.7 g) with implanted jugular catheters were treated by a slow (1-2 min) IV injection with the FPS at the dose of 64 mg/kg on day 0. A group of 4 matched control mice were treated with vehicle (normal saline) alone (group zero, control). Groups of 4 FPS-treated mice were sacrificed at days 1, 5, 10 and 14 (groups 1, 2, 3, and 4, respectively) after FPS treatment (total of 5 groups of 4 mice).

Prior to sacrifice, 100 ul of blood was collected from each animal and analyzed for a complete blood count (CBC) using Hemavet blood count analyzer. Bone marrow was harvested from all mice and red blood cells were lysed using Cell-lysing buffer (Becton Dickinson) according to the manufacturer's directions and cells were washed and filtered through 70 uM cell strainers. Cells were stained according to the following protocol: 1 ug of FcBlock was added to 50 ul aliquots of bone marrow cells and incubated for 10 min on ice. 1 ug of FITC anti mouse CD34 or isotype control was added and samples were incubated on ice for 20 min. Cells were washed twice and resuspended in 50 ul PBS. 3 ul of normal rat serum was added and samples were incubated for 10 min on ice. 20 ul/sample of APC lineage cocktail was added and samples were incubated for 20 min on ice. Cells were washed twice and resuspended in 200 ul PBS for FACS analysis.

Cells were analyzed by flow cytometry on a FACSCalibur flow cytometer. Cells were gated on forward and side scatter parameters expected to include nucleated cells and to exclude any residual red blood cells, platelets or debris. 50,000 gated events per sample were collected. Cells were analyzed using Cell Quest Pro software (Becton Dickinson). Percentages of cells staining with the FITC isotype control were subtracted from the percentages of those stained with FITC anti-CD34 to give a value for specific staining for CD34. Data was analyzed using Students t-test and Prism software (Graphpad Prism Inc.).

### 2.3.2. Results and Discussion.

Bone marrow cells were recovered from limb bones of all animals.

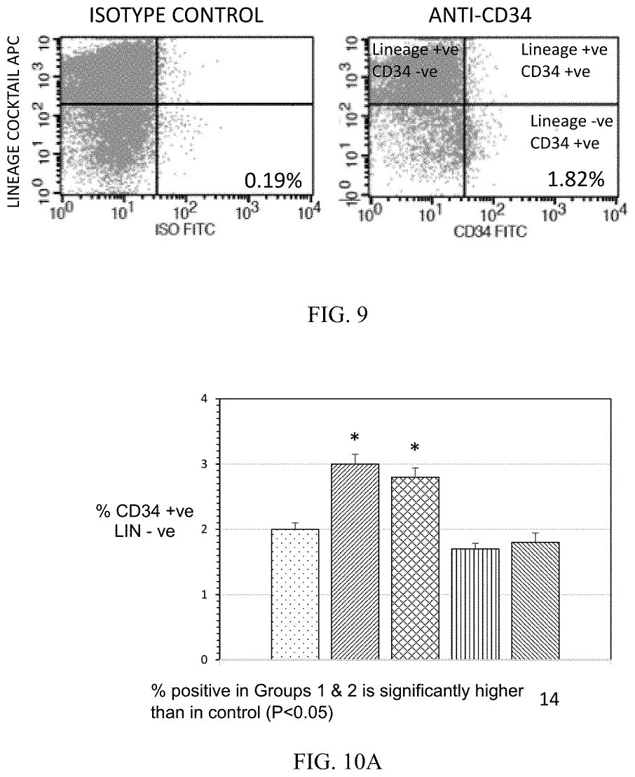

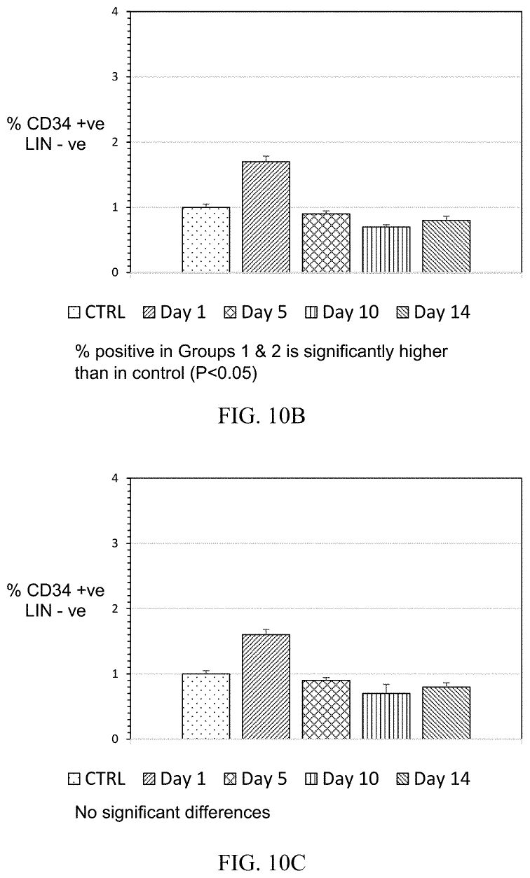

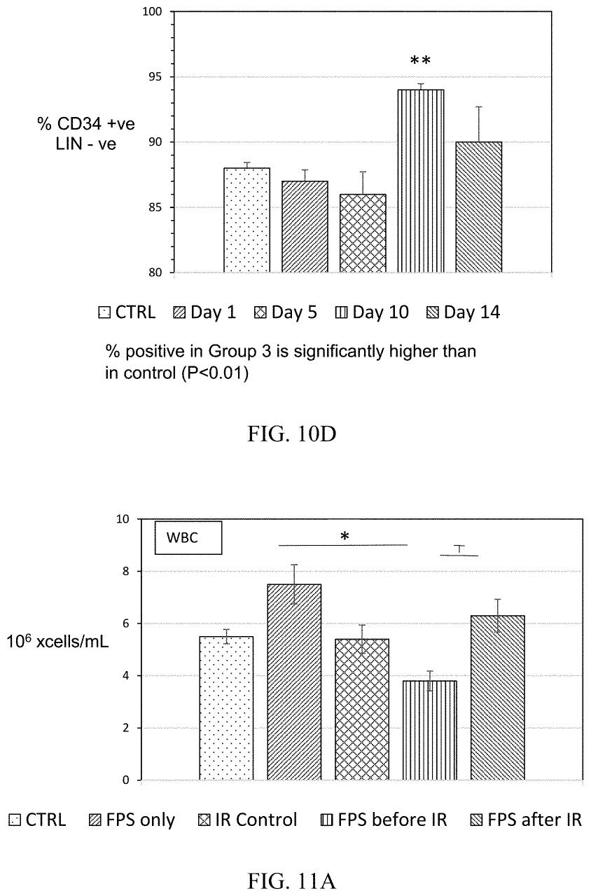

Bone marrow cells were dual-stained with an anti-CD34 antibody and a cocktail of antibodies to various cell lineages and the percentages of subsets of cells positive or negative for the markers was determined as shown in FIGS. 10A-10D.

The data showed that the FPS induced a significant increase in CD34+ve, lineage cocktail −ve cells in the bone marrow at Days 1 and 5 (FIGS. 10A-10D, P<0.05 for each timepoint). Approximately 2% of bone marrow cells were positive for CD34 and negative for lineage cocktail in the bone marrow of control animals. The maximum increase of around 50% was seen on Day 1. By Days 10-14, the percentage of this subset was reduced and was not significantly different from that in the controls (FIGS. 10A-10D).

There was no statistically significant change in the percentage of cells that were positive for both CD34 and lineage markers at any time point, although there did appear to be a small increase in this subset at day 1.

There was a significant increase in the percentage of CD34 −ve, Lineage cocktail +ve cells at day 10 after FPS treatment (P<0.01). At this timepoint, the percentage of CD34+ve lineage cocktail−ve cells in the bone marrow had returned to baseline and it is possible that some of the latter subset had differentiated into lineage positive cells. This is consistent with the statistically significant increase of the monocyte (%) on day 10 (compared to the control levels) and on Day 14 (increase in absolute monocyte counts and % monocytes) in the peripheral blood (Table 11, below). These increases in monocyte percentages and absolute monocyte counts may serve as useful biomarkers in the blood, to indicate the effectiveness of a certain dose of FPS on healthy as well as injured patients.

TABLE 11
Bone Marrow Recoveries

| Group # | Animal # | Cell number × 10⁶ |
|---|---|---|
| 1 | 220 | 26 |
| Day 1 | 225 | 26.2 |
| | 227 | 35.7 |
| | 232 | 29.9 |
| 2 | 221 | 34.3 |
| Day 5 | 228 | 24.2 |
| | 229 | 32.2 |
| | 230 | 35.6 |
| 3 | 223 | 32.4 |
| Day 10 | 231 | 31.8 |
| | 233 | 23.8 |
| | 234 | 31.8 |
| 4 | 214 | 72.4 |
| Day 14 | 218 | 44.2 |
| | 219 | 44.2 |
| CONTROL | 224 | 54.4 |
| | 215 | 35.8 |
| | 217 | 43 |
| | 213 | 46 |

Table 12 below shows the background concentration of monocytes and as a percentage of the white blood cells in peripheral blood.

TABLE 12

| Control | Mouse | Mouse | Mouse | Mean | Standard |
|---|---|---|---|---|---|
| mice (saline) | #213 | #215 | #217 | value | Deviation (SD) |
| Monocyte | 0.33 | 0.36 | 0.27 | 0.32 | 0.05 |
| concentration | | | | | |
| (K/uL) | | | | | |
| Monocyte | 6.34 | 4.62 | 4.21 | 5.06 | 1.13 |
| (% of WBC) | | | | | |

Table 13 shows the increase in one type of white blood cells (monocytes) in peripheral blood after one dose of FPS (64 mg/kg) administered intravenously, known to be enough to stimulate bone marrow CD34 cells, P value (* compare to same mouse background, ** compare to control mice).

TABLE 13

| | BACKGROUND PRIOR TO | | | | | | | | | | | |
|---|---|---|---|---|---|---|---|---|---|---|---|---|
| | FPS | | | | | ONE DAY AFTER FPS DOSE | | | | | P VALUE | |
| GROUP 1 | #220 | #225 | #232 | MEAN | | #220 | #225 | #232 | MEAN | SD | * | ** |
| Monocyte | 0.39 | 0.23 | 0.30 | 0.31 | | 0.27 | 0.36 | 0.42 | 0.35 | 0.08 | 0.42 | 0.58 |
| conc, K/uL | | | | | | | | | | | | |
| Monocyte | 8.78 | 3.44 | 6.41 | 6.21 | | 5.59 | 4.66 | 5.68 | 5.31 | 0.56 | 0.67 | 0.74 |
| (% of | | | | | | | | | | | | |
| WBC) | | | | | | | | | | | | |
| | BACKGROUND PRIOR TO | | | | | | | | | | | |
| | FPS | | | | | FIVE DAYS AFTER FPS DOSE | | | | | P VALUE | |
| GROUP 2 | #221 | #228 | #229 | MEAN | | #221 | #228 | #229 | MEAN | SD | * | ** |
| Monocyte | 0.43 | 0.40 | 0.37 | 0.40 | | 0.26 | 0.31 | 0.23 | 0.27 | 0.04 | 0.029 | 0.205 |
| conc, K/uL | | | | | | | | | | | | |
| Monocyte | 6.06 | 7.32 | 6.94 | 6.77 | | 5.83 | 5.08 | 4.00 | 4.97 | 0.92 | 0.16 | 0.923 |
| (% of | | | | | | | | | | | | |
| WBC) | | | | | | | | | | | | |
| | BACKGROUND PRIOR TO | | | | | | | | | | | |
| | FPS | | | | | TEN DAYS AFTER FPS DOSE | | | | | P VALUE | |
| GROUP 3 | #223 | #231 | #233 | #234 | MEAN | #223 | #231 | #233 | #234 | MEAN | * | ** |
| Monocyte | 0.25 | 0.19 | 0.41 | 0.33 | 0.30 | 0.58 | 0.50 | 0.43 | 0.40 | 0.48 | 0.11 | 0.023 |
| conc, K/uL | | | | | | | | | | | | |
| Monocyte | 3.95 | 3.66 | 4.62 | 5.73 | 4.49 | 6.71 | 6.84 | 5.84 | 6.74 | 6.53 | 0.033 | 0.140 |
| (% of | | | | | | | | | | | | |
| WBC) | | | | | | | | | | | | |

| | | | | | FOURTEEN DAYS AFTER FPS | | | | | | |
|---|---|---|---|---|---|---|---|---|---|---|---|
| | BACKGROUND PRIOR TO FPS | | | | DOSE | | | | | P VALUE | |
| GROUP 4 | #220 | #225 | #232 | MEAN | #218 | #219 | #214 | MEAN | SD | * | ** |

| | | | | | | | |
|---|---|---|---|---|---|---|---|
| Monocyte | Not available | 0.71 | 0.54 | 0.44 | 0.56 | 0.14 | 0.043 |
| conc, K/uL | | | | | | | |
| Monocyte | Not available | 7.37 | 8.40 | 9.43 | 8.40 | 1.03 | 0.019 |
| (% of | | | | | | | |
| WBC) | | | | | | | |

Comments: Control group of mice administered with 8 mL saline per kg weight of the mouse and the FPS-group administered 8 mL (64 mg) per kg had blood drawn at the different time points. The concentrations of various blood cells (thousands per uL of blood) were measured. The various cells studied included: WBC, with subtypes including neutrophils, lymphocytes, monocytes, eosinophils, basophils. These subtypes were expressed also as a % of the WBC in the blood. In addition, RBC, hemoglobin concentration, hematocrit, MCV (mean corpuscular volume), MCH (mean corpuscular hemoglobin), MCHC, RDW, platelet count, MPV (mean platelet volume), ratio of lymphocyte per monocyte (absolute counts); ratio of lymphocyte per monocyte (ratios), inverted ratios of lymphocytes per monocyte were also calculated.

The most interesting finding is the increase in monocyte concentrations as well as % of monocytes in the WBC population. P values were calculated comparing the monocytes changes to either the control animals or the same animal prior to the dosing of FPS. Variations that are significant to the values compared are in red to high light the observation that the increase occurs mainly after day ten (Group 3) and becomes very noticeable on Day 14 after FPS dosing. Because the animals in group 4 did not have their background monocytes measured prior to the dosing of FPS, the values obtained on Day 14 can only be compared to the control mice values. While the control mouse group showed a mean of 0.32 and 5.06 (absolute concentration and % of WBC, respectively), by Day 14, the numbers have increased to 0.56 and 8.40, which are statistically significant (P values of 0.04 and 0.02.

The data is consistent with the finding by Dr. Mao and Yen reported in a Quarterly Report to the NIH for the work done under “Fibrinoplate-S for the Treatment of Radiation-induced Skin Damage” May 2015, and reproduced below for reference. The previous data showed that many types of peripheral blood cells showed remarkable increase on Day 51 after two doses of FPS (8 mg per kg each dose) in rats without irradiation (and similar results in rats post-irradiation.)

In FIGS. 11A-11D, all the dotted columns represent the cell concentrations in the control rats (no irradiation IR, saline). All the right diagonal lined columns represent the result in rats (no irradiation) administered with FPS alone. By Day 51 post-FPS-treatment, several of the cell populations (WBC, lymphocytes, monocytes, granulocytes) showed significant increase compared to the control on Day 51. All the crossed lined columns are results after irradiation (infused with saline): they show concentrations similar to those in the control group (since the irradiation is locally administrated to the hind leg of the rats, to create irradiation-induced skin injury, not a whole-body irradiation which would definitely affect the bone marrow).

The fourth group in the vertically lined columns represents results of “FPS before IR”: this means the FPS was administered as a “prophylactic” drug on Day −2, and −1 before the irradiation event on day 0. The result showed that these cell populations are decreased compared to the control concentrations. The reason is most likely due to the possibility that many stem cells resulting from the FPS stimulation were killed by the irradiation, even though it is a “local” irradiation event.

The fifth group in the left diagonal lined columns represents result of “FPS after IR”. This group seems to have cell concentrations slightly better than the control levels. But these results are still below that of the blue columns, which are the “FPS only” rats. The overall data suggest the FPS has a beneficial effect independent of irradiation (or possibly other injuries). The increase in WBC and its subtypes may be very beneficial to patients who are in need of increased protective cell populations such as in cancer patients who have depleted absolute neutrophil counts after chemotherapy.

### Finding the Effective Dose of FAS to be Used in Severely Irradiated Human Patients

### Introduction

The conventional path of drug evaluation (whether a biological product or defined chemical entity) established by the FDA requires the following steps:
(a) Establishment of the safety and efficacy of the product in animal models,
(b) Establishment of safety in human volunteers (Phase 1)
(c) Establishment of the effective dose in diseased humans (Phase 2)
(d) Establishment of both safety and effectiveness in a large human population which may have other co-morbidities, for the purpose of finding out when not to use the drug (i.e. contraindication.) Phase 3.

However, under certain situations, the conventional path will not be possible. Since Sep. 11, 2001, the FDA has established the “Animal Rule” so that potentially useful treatments for dangerous situations can be evaluated, where it is not ethical to produce the situation on human volunteers, such as would happen after a terrorist attack involving radioactive material and/or toxins. The steps involve the demonstration of safety and efficacy in animal models first where the dangerous or severe degree of harm can be shown to potentially replicate the expected human damage. Then the safety of the drug needs to be demonstrated in healthy human volunteers.

Given the fact that animals can have responses different from humans, an effective dose in the treatment of the animal may not be effective at all in injured humans (after exposure to the irradiation dose or toxin exposure.) Therefore, there exists a need to develop systematic ways to come up with a reasonably scientific method of arriving at a “effective” dose to treat the (future) injured human person under the Animal Rule.

### The Logical Approach:

We will use irradiation damage to the skin as an example of “injured human patient” under the Animal Rule. It is obvious that other injuries can benefit from the same methodology.

In the conventional approach, there are typically 4 populations: (a) non-injured animals; (b) injured animals; (c) non-injured humans; (d) injured humans. If we want to be more detail, we can say there are actually 8 populations when we further divide the above 4 populations into (1) the saline/placebo-treated group, vs (2) the drug-treated group.

Under the Animal Rule, one does not have a standard population of injured humans to study, i.e. population (d), therefore one has to work on only the other 3 populations, regarding the effectiveness of a given dose of drug or treatment.

### Biomarkers.

Since there is no injured humans who can be administered the drug to find out what dose of the drug is effective, one has to work with Biomarkers. Biomarkers are any number of cell markers or cellular signals or cells that can indicate the effect of the drug (with or without injury to the host.)

Examples of biomarkers include: (a) stem cells or progenitor cells in the bone marrow; (b) progenitor cells in the blood; (c) mature cells in the blood. These markers will need to be demonstrated in the non-injured animals as well as healthy human volunteers first. Then their pattern in the injured animals will provide hints as to how a dose of the drug will affect the injured (future) human patient.

An example of (a) will be the study of progenitor cells in the bone marrow mentioned above. An example of (c) will be the data published by Mao et al. in 2015 in the Radiation Research on the higher concentration of white blood cells in the blood of the FAS-treated rats without irradiation.

### Detail Discussion of how to Relate Animal Data to Potentially Acceptable Treatment in Humans:

Since the gold standard of treatment is the actual healing of the wound (or curing of the disease state) one needs data on the macroscopic scale of observing the healing process after a dose of the drug.

The first step is to find the Effective Dose in the injured animal. An example of this is the discovery that a dose of 8 mg/kg (even when dosed twice, and on different days with respect to the day of irradiation) in the rat is effective in healing the irradiation damage on the skin caused by a local beam at 25 Gy.

The second step is to find the Tolerated Dose, so that if a higher dose is needed (whether in the animal model or in non-injured humans) it can be used. Data obtained in a non-irradiated, non-injured mouse model showed that a 7-day continuous infusion of FPS (via an implanted port) at a rate of 5 mL per kg per hour (highest tolerated rate of fluid infusion) can be tolerated. These mice showed no clinical signs and the hematological data as well as clinical chemistry collected 1 day after completion of infusion of FPS showed no adverse effects.

Since the concentration of FAS is 8 mg per mL in the drug product FPS, the data means a total dose of 5×8 mg/kg/hr×24 hr×7 days is well tolerated in mice. This total dose of 6,720 mg/kg of FAS in mice is 840 times the effective dose in rats. However, the toxicologist typically counts only a “daily dose” meaning the daily dose in the mice is only 5×8×24, or 960 mg per kg, which is still 120 times that of the effective dose in the rat.

However, the mice has a surface-to-body-mass ratio of 12 times higher than the human body—so a further discount is needed. The tolerated dose expected in the human is to have 960 mg/kg divided further (artificially) by 12. This means the tolerated human dose is at least 80 mg per kg, which is still higher than the expected “Effective Dose” of 8 mg per kg (if the irradiated human has the same response as the irradiated rat.)

For the discussion below we will assume that the standard effective dose is 8 mg per kg, even though a higher dose of FAS may be used (mainly to elicit a response in the Biomarker). We will also assume that an irradiation dose of 25 Gy is representative of irradiation damage, although it is clear that higher doses can cause more damage, up to a point where no amount of administered FAS can heal the wound (or prevent mortality of the animal.)

In less severe cases, such cancer patients receiving radiation therapy, it is recognized that a dose of 1 Gy will already lead to injury, and higher doses can be fatal.

### Detail Discussion of how to Use Biomarkers to Establish a Reliable Effective Dose of FAS for the Injured Human Patient:

Work needs to be done in the animal model to show whether it is really necessary to give 2 doses of FAS at 8 mg per kg, or whether 1 dose is sufficient. Work also needs to be done to show that the “delayed” administration of FAS is still beneficial, e.g. after the day of irradiation on day 0, will the administration of one dose of FAS, given on day 1, or 5, or 10, or 15 be still effective in terms of healing the skin lesion which will appear typically 1 month later? This is important because not all irradiated patients can show up within one or two days after the irradiation event. In the situation where the irradiation is due to an atomic event, health care facilities may not be available within a reasonable time at all.

Table 14 assumes that the “best” biomarker were located to be used. Pilot experiments needs to be done in non-injured animals to show which biomarker will show up early (post-FPS-administration), easy to measure, and reliable.

It will be noted that in the unfortunate event that a nuclear incident does occur, obviously some humans will have been irradiated. Such irradiated humans will not be good subjects for the administration of FAS if the effective dose for the potential injured human subject has not been previously established. The reasons are: (a) in the absence of previously approved used established under the Animal Rule, there is no approved use for humans; (b) these already irradiated humans most likely would have suffered co-morbidity such as burns, fractures, lacerations and contamination from radioactive or chemical entities in the environment. Therefore, the collection of data in these injured human victims under any drug treatment will not generate meaning scientific data.

Table 14 below shows 4 populations of which only 3 are available for study.

Population I is the non-irradiated animals; II is the irradiated animals; III is the non-irradiate healthy human volunteer. Population IV is not available.

The steps are as follows:

Step 1: work with the non-irradiated animals first: give an ascending dose of FPS (from 4, to 10 to 40 mL per kg) and measure the Biomarker on the most optimal day (which has been established in pilot studies, not shown in Table 14). The control non-irradiated animals received an equivalent dose of normal saline (at 4, 10, 40 mL per kg, over a period of time tolerated by the animal species.) It is noted that the effective dose to heal a skin lesion is 8 mg per kg, which is only 1 mL of FPS per kg. Therefore, this regiment represents a very high dose, for the purpose of provoking a strong response from the non-irradiated animal.

The last row (Hypothetical Biomarker value) provided a hypothetical value to the response of the animal. It can be seen that normal saline does not increase the value of the response, while FPS provides a response up to 0.6 units.

The ratio between the FPS group vs the Saline group which is the highest (0.6 vs 0.1) is obtained when a dose of FSP of 10 mL per kg is administered. Even though 40 mL of FPS will also provoke a response of 0.6 units, further studies will show that this higher dose of 40 mL per kg of FPS may not be necessary.

Step 2: repeat the work in Population II, which are the irradiated animals, which have been irradiated with the standard dose of irradiation.

The hypothetical value of the response indicated in the Saline treated group of irradiated animals, there is a response to the stimulus from the injury. The value in the pre-NS-treatment (after irradiation) is already 0.3; thereafter rising slightly to 0.4—which means the response is mainly to the irradiation and not to the saline administration.

Among FPS-treated animals in Population II (irradiated), the Biomarker value increased up to 0.9, meaning that the FPS administration further increased the response of the Biomarker.

Again, the biggest differential between the FPS-treated vs Saline-treated in all these irradiated animals (0.9 vs 0.4) is provided when 10 mL of FPS per kg is administered.

TABLE 14
Work to be done to find out Effective Dose for Irradiated Humans (not available now)

| | ANIMALS | | | | | | | | | | | | | | | |
|---|---|---|---|---|---|---|---|---|---|---|---|---|---|---|---|---|
| | POPULATION I: NON-IRRADIATED | | | | | | | | POPULATION II: IRRADIATED | | | | | | | |
| | (day 0) | | | | | | | | (on day 0) | | | | | | | |
| | NS-TREATED | | | | FPS-TREATED | | | | NS-TREATED | | | | FPS-TREATED | | | |
| | (on day 1) | | | | (on day 1) | | | | (on day 1) | | | | (on day 1) | | | |
| | | 4 | 10 | 40 | | 4 | 10 | 40 | | 4 | 10 | 40 | | 4 | 10 | 40 |
| | Pre- | mL/ | mL/ | mL/ | Pre- | mL/ | mL/ | mL/ | Pre- | mL/ | mL/ | mL/ | Pre- | mL/ | mL/ | mL/ |
| | NS | kg | kg | kg | FPS | kg | kg | kg | NS | kg | kg | kg | FPS | kg | kg | kg |
| Hypothetical | 0.1 | 0.1 | 0.1 | 0.1 | 0.1 | 0.5 | 0.6 | 0.6 | 0.3 | 0.4 | 0.4 | 0.4 | 0.3 | 0.7 | 0.9 | 0.9 |
| Biomarker | | | | | | | | | | | | | | | | |
| value | | | | | | | | | | | | | | | | |
| | HUMANS | | | | | | | | | | | | | | | |
| | POPULATION III: | | | | | | | | POPULATION IV: not-available | | | | | | | |
| | NON-IRRADIATED | | | | | | | | (hypothetical at this time) IRRADIATED | | | | | | | |
| | (day 0) | | | | | | | | (on day 0) | | | | | | | |
| | NS-TREATED | | | | FPS-TREATED | | | | NS-TREATED | | | | FPS-TREATED | | | |
| | (on day 1) | | | | (on day 1) | | | | (on day 1) | | | | (on day 1) | | | |
| | | 4 | 10 | 40 | | 4 | 10 | 40 | | 4 | 10 | 40 | | 4 | 10 | 40 |
| | Pre- | mL/ | mL/ | mL/ | Pre- | mL/ | mL/ | mL/ | Pre- | mL/ | mL/ | mL/ | Pre- | mL/ | mL/ | mL/ |
| | NS | kg | kg | kg | FPS | kg | kg | kg | NS | kg | kg | kg | FPS | kg | kg | kg |
| Hypothetical | 0.2 | 0.2 | 0.2 | 0.2 | 0.2 | 0.8 | 1.0 | 1.0 | | | | | | | | |
| Biomarker | | | | | | | | | | | | | | | | |
| value | | | | | | | | | | | | | | | | |

Since independent studies on the Effective Dose of FPS on healing the skin lesion has been established to be 1 mL per kg, it is reasonable to designate 10 mL per kg as the potential Effective Dose in the future irradiated human patient.

Step 3: work on healthy human volunteers. Blood will be collected pre-treatment and on the most optimal day (established in animal studies, or further established from multiple-day sampling in human volunteers) for assay of the same Biomarker. Human volunteers will be dosed with 4, 10, 40 mL of FPS per kg. The Table 14 states “day 1” to be consistent with the other populations, but since there is no irradiation, any day can be used for FPS dosing.

The hypothetic data shows that the FPS of 10 mL per kg is again the dose provoking the biggest differential between the FPS group vs the Saline group (1.0 vs 0.2 in the value of the Biomarker).

Conclusion: even though Population IV is not available, the scientific data show that the dose of FPS at 10 mL per kg (80 mg per kg) is likely to provoke a Biomarker response indicative of a healing response in the future irradiated human patient. Since this dose has been shown to be safe in human non-irradiated populations, and safe and effective in animal models, this dose can be accepted as the effective dose to be used in the future on highly irradiated patients.

On patients who potentially can receive skin damage who have been irradiated with lower doses of irradiation (such as done in radiation therapy) the standard FDA-approval approach can be used, since there are plenty of cancer patients who can be enrolled to study how FPS can be efficacious when used properly to this population.

### Further Conclusions are as Follows:

It has been found according to one or more aspects of the present technology that the administration of nanospheres such as FAS to a healthy subject not suffering from any injury will respond to the administration of the nanospheres by having increased concentrations of certain biological markers (biomarkers) where the biomarkers can be mature cells, progenitor cells, stem cells, cell-communication signals such as cytokines, cells with altered oxidative/reductive states, or intra-bone-marrow cells.

It has been found according to one or more aspects of the present technology that the administration of nanospheres to a healthy subject not suffering from any injury will respond to the administration of the nanospheres such as FAS by having increased concentrations of certain biological markers (biomarkers) where the biomarkers are located at their site of origin such as the bone marrow, or endothelium; or at the site of transit from the site of origin to the site of destination, such as blood; or at the site of destination such as an organ like the skin or liver, or a tissue such as muscle or blood vessels.

It has been found according to one or more aspects of the present technology that the administration of nanospheres such as FAS to a healthy subject not suffering from any injury will respond to the administration of the nanospheres by having increased concentrations of certain biological markers (biomarkers) the concentrations of which can reliably predict the dose of nanospheres which will be effective in the treatment of human beings who may suffer from future injuries where the injuries are irradiation-induced.

It has been found according to one or more aspects of the present technology that the administration of nanospheres to a healthy subject not suffering from any injury will respond to the administration of the nanospheres by having increased concentrations of certain biological markers (biomarkers) the concentrations of which can reliably predict the dose of nanospheres which will be effective in the treatment of human beings who may suffer from future injuries where the injuries are caused by polytrauma.

Therefore, the foregoing is considered as illustrative only of the principles of the present technology. Further, since numerous modifications and changes will readily occur to those skilled in the art, it is not desired to limit the present technology to the exact construction and operation shown and described, and accordingly, all suitable modifications and equivalents may be resorted to, falling within the scope of the present technology.

### REFERENCES

1. Gieringer M, Gosepath J, Naim R. Radiotherapy and wound healing: Principles, management and prospects (review). Oncol Rep. 2011; 26(2):299-307. doi:10.3892/or.2011.1319
2. Radiation-Induced Skin Injury: A Challenging Issue in the Outpatient Wound Clinic|Today's Wound Clinic. Accessed Jul. 29, 2020.
3. Jacobson L K, Johnson M B, Dedhia R D, Niknam-Bienia S, Wong A K. Impaired wound healing after radiation therapy: A systematic review of pathogenesis and treatment. JPRAS Open. 2017; 13:92-105. doi:10.1016/jjpra.2017.04.001
4. Wound Healing in PatientsWith Cancer. Accessed Jul. 10, 2020.
5. Dormand E L, Banwell P E, Goodacre T E E. Radiotherapy and wound healing. Int Wound J. 2005; 2(2):112-127. doi:10.1111/j.1742-4801.2005.00079.x
6. Boyce D E, Shokrollahi K. Reconstructive surgery The “reconstructive ladder.” BMJ. 2006; 332(7543):710-712. doi:10.1136/bmj.332.7543.710
7. So W, Wong I, Lee D, et al. Effect of Compression Bandaging on Wound Healing and Psychosocial Outcomes in Older People with Venous Ulcers: A Randomised Controlled

Trial. Vol 20.; 2014. Accessed Jul. 28, 2020.
8. Comerota A J. Intermittent pneumatic compression: Physiologic and clinical basis to improve management of venous leg ulcers. J Vasc Surg. 2011; 53(4):1121-1129. doi:10.1016/j.jvs.2010.08.059
9. Labler L, Rancan M, Mica L, Harter L, Mihic-Probst D, Keel M. Vacuum-assisted closure therapy increases local interleukin-8 and vascular endothelial growth factor levels in traumatic wounds. J Trauma—Inj Infect Crit Care. 2009; 66(3):749-757. doi:10.1097/TA.0b013e318171971a
10. Bhutani S, Vishwanath G. Hyperbaric oxygen and wound healing. Indian J Plast Surg. 2012; 45(2):316-324. doi:10.4103/0970-0358.101309
11. Enoch S, Grey J E, Harding K G. Non-surgical and drug treatments. Br Med J. 2006; 332(7546):900-903. doi:10.1136/bmj.332.7546.900
12. Ahmadi M, Khalili H. Potential benefits of pentoxifylline on wound healing. Expert Rev Clin Pharmacol. 2016; 9(1):129-142. doi:10.1586/17512433.2016.1109443
13. Wang A S, Armstrong E J, Armstrong A W. Corticosteroids and wound healing: Clinical considerations in the perioperative period. Am J Surg. 2013; 206(3):410-417. doi:10.1016/j.amjsurg.2012.11.018
14. Payne W G, Naidu D K, Wheeler C K, et al. Wound healing in patients with cancer. Eplasty. 2008; 8:e9. Accessed Jul. 28, 2020.
15. Flores-Balcázar CH, Flores-Luna L, Villarreal-Garza C, Mota-Garcia A, Bargallo-Rocha E. Impact of Delayed Adjuvant Radiotherapy in the Survival of Women with Breast Cancer. Cureus. 2018; 10(7). doi:10.7759/cureus.3071
16. Dormand E L, Banwell P E, Goodacre T E E. Radiotherapy and wound healing. Int Wound J. 2005; 2(2):112-127. doi:10.1111/j.1742-4801.2005.00079.x
17. The role of cytokines in the development of normal-tissue reactions after radiotherapy—PubMed. Accessed Jul. 23, 2020.
18. Shi H P, Efron D T, Most D, Barbul A. The role of iNOS in wound healing. Surgery. 2001; 130(2):225-229. doi:10.1067/msy.2001.115837
19. Differential expression of inflammatory mediators in radiation-impaired wound healing—PubMed. Accessed Jul. 23, 2020.
20. Effects of radiation on wound healing—PubMed. Accessed Jul. 23, 2020.
21. Lu G, Ling K, Zhao P, et al. A novel in situ-formed hydrogel wound dressing by the photocross-linking of a chitosan derivative. Wound Repair Regen. 2010; 18(1):70-79. doi:10.1111/j.1524-475X.2009.00557.x
22. Samady L, Dennis J, Budhram-Mahadeo V, Latchman D S. Activation of CDK4 gene expression in human breast cancer cells by the Brn-3b POU family transcription factor. Cancer Biol Ther. 2004; 3(3):317-323. doi:10.4161/cbt.3.3.698
23. Sugiyama K, Ishii G, Ochiai A, Esumi H. Improvement of the breaking strength of wound by combined treatment with recombinant human G-CSF, recombinant human M-CSF, and a TGF-β1 receptor kinase inhibitor in rat skin. Cancer Sci. 2008; 99(5):1021-1028. doi:10.1111/j.1349-7006.2008.00761.x
24. Rigotti G, Marchi A, Gali?? M, et al. Clinical Treatment of Radiotherapy Tissue Damage by Lipoaspirate Transplant: A Healing Process Mediated by Adipose-Derived Adult Stem Cells. Plast Reconstr Surg. 2007; 119(5):1409-1422. doi:10.1097/01.prs.0000256047.47909.71
25. Kim W S, Park B S, Sung J H. The wound-healing and antioxidant effects of adipose-derived stem cells. Expert Opin Biol Ther. 2009; 9(7):879-887. doi:10.1517/14712590903039684
26. Kim W S, Park B S, Park S H, Kim H K, Sung J H. Antiwrinkle effect of adipose-derived stem cell: Activation of dermal fibroblast by secretory factors. J Dermatol Sci. 2009; 53(2):96-102. doi:10.1016/j.jdermsci.2008.08.007
27. Kim J-H, Jung M, Kim H-S, Kim Y-M, Choi E-H. Adipose-derived stem cells as a new therapeutic modality for ageing skin. Exp Dermatol. 2011; 20(5):383-387. doi:10.1111/j.1600-0625.2010.01221.x
28. Kim J Y, Suh W. Stem cell therapy for dermal wound healing. Int J Stem Cells. 2010; 3(1):29-31. doi:10.15283/ijsc0.2010.3.1.29
29. Asahara T, Masuda H, Takahashi T, et al. Bone marrow origin of endothelial progenitor cells responsible for postnatal vasculogenesis in physiological and pathological neovascularization. Circ Res. 1999; 85(3):221-228. doi:10.1161/01.RES0.85.3.221
30. Cancer Supportive Care Drugs Market Size|Industry Report, 2019-2026.

Accessed Jul. 15, 2020.
31. Cancer Supportive Care Drugs Market|Growth, Trends, and Forecasts (2020-2025). Accessed Aug. 6, 2020.
32. Global Cancer Data 2018—WHO; 2018. Accessed Aug. 6, 2020.
33. Cancer Statistics—National Cancer Institute. Accessed Jul. 29, 2020.
34. Macià i Garau M, Lucas Calduch A, López EC. Radiobiology of the acute radiation syndrome. Reports Pract Oncol Radiother. 2011; 16(4):123-130. doi:10.1016/j.rpor.2011.06.001
35. Bryan W. Top Ten 2017 NASA iTech Finalists Announced. Published online 2017. Accessed Jul. 29, 2020.
36. Sung A D, Yen R, Deoliveira D, et al. Fibrinogen-Coated Nanospheres Prevent Thrombocytopenia-Related Bleeding.; 2015. doi:10.1016/j.bbmt.2014.11.140
37. Yen R C K. US20170128545A9—Albumin nanoparticles to augment stem cell function in vivo. Accessed Jul. 29, 2020.
38. Tilling L, Chowienczyk P, Clapp B. Progenitors in motion: Mechanisms of mobilization of endothelial progenitor cells. Br J Clin Pharmacol. 2009; 68(4):484-492. doi: 10.1111/j.1365-2125.2009.03486.x
39. Corbel C, Vaigot P, Salaün J. αIIb integrin, a novel marker for hemopoietic progenitor cells. Int J Dev Biol. 2005; 49(2-3 SPEC. ISS.):279-284. doi:10.1387/ijdb.041936cc
40. Asahara T, Masuda H, Takahashi T, et al. Bone marrow origin of endothelial progenitor cells responsible for postnatal vasculogenesis in physiological and pathological neovascularization. Circ Res. 1999; 85(3):221-228. doi:10.1161/01.RES.85.3.221
41. Ryan J L, Heckler C E, Ling M, et al. Curcumin for radiation dermatitis: A randomized, double-blind, placebo-controlled clinical trial of thirty breast cancer patients. Radiat Res. 2013; 180(1):34-43. doi:10.1667/RR3255.1
42. Katsuta E, Oshi M, Rashid O M, Takabe K. Generating a murine orthotopic metastatic breast cancer model and performing murine radical mastectomy. J Vis Exp. 2018; 2018(141). doi:10.3791/57849
43. Poleszczuk J, Luddy K, Chen L, et al. Neoadjuvant radiotherapy of early-stage breast cancer and long-term disease-free survival. Breast Cancer Res. 2017; 19(1):75. doi:10.1186/s13058-017-0870-1
44. Sousa C, Cruz M, Neto A, et al. Neoadjuvant radiotherapy in the approach of locally advanced breast cancer. ESMO Open. 2020; 4(2). doi:10.1136/esmoopen-2019-000640
45. Housman G, Byler S, Heerboth S, et al. Drug resistance in cancer: An overview. Cancers (Basel). 2014; 6(3):1769-1792. doi:10.3390/cancers6031769
46. Rashid O M, Nagahashi M, Ramachandran S, et al. An improved syngeneic orthotopic murine model of human breast cancer progression. Breast Cancer Res Treat. 2014; 147(3):501-512. doi:10.1007/s10549-014-3118-0
47. Rodriguez J J, Kung T, Wang Y, et al. Changes in skin vascularity in a murine model for postmastectomy radiation. In: Annals of Plastic Surgery. Vol 76. Lippincott Williams and Wilkins; 2016:494-498. doi:10.1097/SAP.0000000000000628
48. Aoki H, Aoki M, Yang J, et al. Murine model of long-term obstructive jaundice. J Surg Res. 2016; 206(1):118-125. doi:10.1016/j.jss.2016.07.020

What is claimed as being new and desired to be protected by Letters Patent of the United States is as follows:

1. A method of treating multiple traumas in a subject in need thereof, the method comprising administering a therapeutically effective amount of a protein nanosphere suspension containing submicron albumin spheres to the subject suffering from multiple types of traumas (polytrauma) at a dose sufficient to increase a survival rate of the subject and decrease a morbidity from any one of the traumas, the albumin spheres being configured to augment a function or effectiveness of stem cells or precursor cells in vivo to stimulate mobilization of the stem cells or the precursor cells toward the traumas, wherein the traumas are different from each other.

2. The method of claim 1, wherein the subject is human.

3. The method of claim 1, wherein the therapeutically effective amount is 8 mg/kg or more administered to the subject intravenously.

4. The method of claim 1 further comprising the step of stimulating a conversion of the stem cells or precursor cells to mature cells.

5. The method of claim 1, wherein the albumin spheres of the albumin nanoparticle suspension are bound with fibrinogen molecules to produce fibrinogen-coated albumin nano-spheres (FAS).

6. The method of claim 1, wherein the albumin nanoparticle suspension includes a supernatant solution configured to maintain osmolarity compatible with blood of the subject, wherein the supernatant is saline.

7. The method of claim 1, wherein the multiple types of traumas is selected from the group consisting of any one or any combination of a surgical wound, an irradiation burn, a burn, a blunt trauma, a bone injury, a chemotherapy agent, a poison or toxin, and an irradiation dose.

8. The method of claim 1, wherein the multiple types of traumas is a surgical wound and an irradiation injury, wherein the surgical wound is before or after the irradiation wound.

9. The method of claim 1 further comprises the step of increasing concentrations of one or more biological markers (biomarkers) by the albumin spheres, wherein the biomarkers are selected from the group consisting of any one or any combination of the stem cells, a second stem cell different to that of the stem cells, mature cells, progenitor cells, cell-communication signals, cytokines, cells with altered oxidative/reductive states, and intra-bone-marrow cells.

10. The method of claim 9, wherein the biomarkers are located at a site selected from the group consisting of a) an origin site being bone marrow or endothelium, b) a transit site from the origin site to a destination site being blood, an organ, a muscle or blood vessels, and c) a destination site which includes blood, an organ, a muscle or blood vessels.

11. The method of claim 10, wherein the concentrations of the biomarkers are configured to predict a dose of the submicron albumin spheres which is effective in a further treatment from future injuries caused by the multiple traumas.

12. The method of claim 1, wherein the submicron albumin spheres increases CD34 cells in bone marrow of the subject.

13. The method of claim 1, wherein the albumin spheres are fibrinogen-coated albumin nano-spheres (FAS) that further ameliorates an inflammatory response of the subject by decreasing an amount of Regulated upon Activation, Normal T Cell Expressed and Presumably Secreted (RANTES) endothelial production on cytokines production, or improves a secretion of fractalkine (FKN).

14. A composition for treating multiple traumas in a subject in need thereof, the composition comprising a therapeutically effective amount of an albumin nanoparticle suspension containing submicron albumin spheres, the albumin spheres being configured to augment a function or effectiveness of stem cells or precursor cells in vivo to stimulate mobilization of the stem cells or the precursor cells toward the traumas, wherein the traumas are different from each other.

15. The composition of claim 14 wherein the subject is human.

16. The composition of claim 14, wherein the multiple types of traumas is selected from the group consisting of any one or any combination of a surgical wound, an irradiation burn, a burn, a blunt trauma, a bone injury, a chemotherapy agent, a poison, and an irradiation dose.

17. The composition of claim 14, wherein the albumin spheres of the albumin nanoparticle suspension are bound with fibrinogen molecules to produce fibrinogen albumin spheres (FAS).

18. The composition of claim 14, wherein the albumin nanoparticle suspension includes a supernatant configured to maintain osmolarity compatible with blood of the subject, and wherein the supernatant is saline added.

19. The composition of claim 14, wherein the albumin spheres of the albumin nanoparticle suspension are further configured to increase concentrations of one or more biological markers (biomarkers) selected from the group consisting of any one or any combination of the stem cells, a second stem cell different to that of the stem cells, mature cells, progenitor cells, cell-communication signals, cytokines, cells with altered oxidative/reductive states, and intra-bone-marrow cells.

20. The composition of claim 14, wherein the albumin spheres are fibrinogen-coated albumin nano-spheres (FAS) that are further configured to ameliorate an inflammatory response of the subject by decreasing an amount of Regulated upon Activation, Normal T Cell Expressed and Presumably Secreted (RANTES) endothelial production on cytokines production, or improves a secretion of fractalkine (FKN).
